(19) United States
(12) Patent Application Publication
(10) Pub. No.: US 2006/0095982 A1
(43) Pub. Date: May 4, 2006

(54) Phytoremediation of contaminant compounds via chloroplast genetic engineering

(75) Inventor: Henry Daniell, Winter Park, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Central Florida, Orlando, FL (US)

(21) Appl. No.: 10/520,204

(22) PCT Filed: Jul. 2, 2003
(86) PCT No.: PCT/US03/20868

§ 371 Date: Aug. 26, 2005

Related U.S. Application Data
(60) Provisional application No. 60/393,451, filed on Jul. 3, 2002.

(51) Int. Cl. A01H 1/00; C12N 15/82

(52) U.S. Cl. 800/278; 435/468

(57) ABSTRACT

A plastid transformation vector for stably transforming a plastid genome, comprising, as operably-linked components, a first flanking sequence, at least one DNA sequence coding for a polypeptide suitable for remediating a contaminant compound, and a second flanking sequence, wherein a plant is stably transformed with the plastid transformation vector, and the plant is capable of phytoremediating a contaminant compound.

### FIELD OF THE INVENTION

This application relates to the field of genetic engineering of plant plastid genomes, particularly chloroplast, vectors for transforming plastids, transformed plants, progeny of transformed plants, and to methods for transforming plastid genomes and plants to generate express genes which are suitable to bioremediate contaminant compounds. This application further relates to plastid genetic engineering to enhance the capacity of plants for phytoremediation. Further, one aspect of this application relates to integrating a native operon containing the phytoremediation genes (without any codon modification), which code for a contaminant reductase enzyme capable of breaking down its respective contaminant, and wherein the operon is introduced into the vector through a single transformation event.

### BACKGROUND

All references cited to in the background section of this specification are incorporated into this application by reference, as are all references and publications reffered to throughtout this application.

Global industrialization has resulted in the release of large amounts of potentially toxic compounds into the biosphere, among them trace elements like arsenic, cadmium, lead, mercury and nickel, which are commonly addressed as heavy metals (Nriagu 1979; Nieboer and Richardson 1980; Nriagu and Pacyna 1988). The use of plants to clean-up soils contaminant with trace elements could provide a cheap and sustainable technology for bioremediation. However, at the current state a number of field trials suggested that the rate of contaminant removal using conventional plants and growth conditions is insufficient. Consequently the art recognized that the introduction of novel traits into high biomass plants in a transgenic approach was a promising strategy for the development of effective phytoremediation technologies.

This has been exemplified by generating plants able to convert organic and ionic forms of mercury into the less toxic, volatile, elemental mercury, a trait that occurs naturally. To date however, the levels of bioremediation transgenes expressed n plants has been to low to act as a suitable source for the remedeation of contaminant compounds.

Whereas preventing the release of toxins into the environment should be the primary objective, large areas of soils world-wide still remain polluted with toxic levels of trace elements. Cleaning-up most of these soils is necessary in order to reclaim the areas and to minimize the entry of potentially toxic elements into the food chain. A large-scale program for environmental clean-up has been initiated in the United States

The concept of phytoremediation—using plants to remove or inactivate pollutants from soils and surface waters has received increasing attention in recent years; and it has been the subject of a number of reviews (Chaney 1983; Cunningham et al. 1995; Salt et al. 1995, 1998; Raskin 1996; Chaney et al. 1997; Raskin et al. 1997; Blaylock and Huang 2000; Kärenlampi et al. 2000; Meagher 2000; Pilon-Smits and Pilon 2000). Still and despite these studies, no one do date has sucessfully transformed the plastid genome to express contaminant reducing proteins at levels sufficient to fully bioremediate contaminant compounds.

Compared to existing physical and chemical methods of soil remediation, for example excavation and burial, the use of plants is cost-effective and less disruptive to the environment (Cunningham and Berti 2000). The global phytoremediation market of approximately U.S. $ 35 million in 1999 has been predicted to grow ten-fold over the coming 5 years. It has good chances in the future to increase its share of the global remediation market, which was approximately U.S. $ 18-19 billion in 1998 (Glass 1999).

Improvement of plants by genetic engineering offers a new possibility for upgrading phytoremediation technology. Introducing foreign genes to the plants could alter, for example, metal uptake, transport and accumulation as well as metal tolerance in the plants (Kärenlampi et al., 2000). Recently, genetically engineered plants have improved to be used as “bio-converters” of highly toxic forms of some metal to less toxic and/or volatile forms that is subsequently released into the atmosphere. One of the most exciting mechanisms in phytoremediation is the “phytovolatilization” in which some elements (e.g. mercury and selenium) can exist in a variety of states, including different cationic and oxyanionic species and thio- and organo-metallics offers exciting phytoremediation possibilities by transformation of toxic forms into relatively less toxic and volatilized forms. For example, it was estimated that 10% to 30% of the removed selenium was accumulated by different plant species was removed by phytovolatilization (Terry et al., 1992, Pilon-Smits et al., 1999), and these processes make them excellent candidates for the phytoremediation of Se-contaminated sites (Terry and Zayed, 1994, 1998, Zayed and Terry, 1994, Banuelos et al., 1995).

Plants use photosynthetic energy to extract ions from the soil and to concentrate them in their biomass, according to nutritional requirements. When present at elevated levels, those contaminants which are essential or non-essential trace elements are able to enter plants by virtue of their chemical similarity to (other) nutrients ions. For example, AsO4 3—or Cd2+ (Lane and Morel 2000) can enter roots through the uptake systems for PO4 3—or Fe2+/Ca2+, respectively (Meharg and Macnair 1992; Clemens et al. 1998; Cohen et al. 1998). The aim of phytoextraction is to harness the nutrient acquisition system of plants in order to achieve maximum accumulation of pollutant trace elements in the above-ground tissues. Above-ground biomass is then harvested, thereby removing the pollutant from the site in a small number of successive growth periods. Plant material can be ashed to further concentrate the pollutant, and then possibly be recycled in metal smelting, or deposited in specialized dumps. When grown at a contaminant site a plant used in phytoextraction is required: (1) to accumulate large amounts of one or several trace elements in the shoot, (2) to exhibit a high rate of biomass production and (3) to develop an extensive root system. Further, the chemical properties of a small number of pollutant trace elements, mainly mercury and selenium, allow the use of the technology of phytovolatilization. Instead of accumulating inside the plant, the trace element is enzymatically transformed into a less toxic, volatile compound and is subsequently released into the atmosphere (Rugh et al. 1996; Meagher 2000; Pilon-Smits and Pilon 2000).

A particularly well studied and characterized pollutant is Mercury (Hg). Hg pollution of soil and water is a world-wide problem (Dean et al., 1972; Kramer and Chardonnens, 2001). The extent to which Hg is harmful depends on the form of mercury present in the ecosystem. Inorganic forms of Hg are less harmful than organic forms partly because they bind strongly to the organic components of soil. For this reason, Hg does not tend to contaminant the ground water except when Hg leaches from a municipal landfill (USEPA, 1984). Organomercurial compounds on the other hand, may be 200 times more toxic than inorganic Hg (Patra and Sharma, 2000) and methyl-Hg is especially toxic (Meagher and Rugh, 1997).

The principal forms of organomercurial compounds are alkyl mercurials (methyl- and ethyl-Hg), aryl mercurials (phenyl-Hg), and alkoxy alkyl Hg diuretics. The excessive use of organomercurial compounds (e.g., in fertilizers and pesticides) is known to have severe effects on plants. The main site of action of Hg damage appears to be the chloroplast thylakoid membranes and photosynthesis. Organomercurial compounds have been shown to strongly inhibit electron transport, oxygen evolution (Bernier et al., 1993), Hill reaction, photophosphorylation and to quench chlorophyll fluorescence in photosystem II (Kupper et al., 1996). Furthermore, Prasad and Prasad, 1987, showed that Hg might replace Mg from the chlorophyll moiety leading to a reduction in chlorophyll content. Sen and Mondal (1987) and Sinha et al. (1996) reported a 26% reduction of chlorophyll content in Salvia natans, and 35% in Bacopa monnieri at 5 μg/ml HgCl₂, even though these plants have a natural tolerance to Hg.

Current remediation methods to clean up heavy metal-contaminant soils include soil flushing, chemical reduction/oxidation and excavation, retrieval and offsite disposal, all of which are expensive, environmentally invasive, and labor intensive (Karenlampi et al., 2000). An alternative and more cost-effective approach is phytoremediation, i.e., the use of plants to clean up contaminant environments (Lin et al., 1995; Salt et al., 1995; Terry et al., 2000). With the aid of genetic engineering, plants can be genetically modified to substantially improve phytoremediation. Expression of several plant and bacterial genes in transgenic plants has significantly enhanced these plant remediation systems (Meagher, 2000; Doucleff and Terry, 2002). Several studies have successfully integrated bacterial genes into nuclear genomes to produce plants that were specifically engineered for phytoremediation of metal-polluted environments (Heaton et al, 1998; Rugh et al., 1998; Nies, 1999). With respect to Hg, plants have been engineered with modified bacterial mercuric ion reductase (merA) and organomercurial lyase (merB) genes; these enzymes are capable of converting highly toxic methylHg into the much less toxic Hg(0), which may then be volatilized (Rugh et al., 1996, Bizily et al., 1999, 2000).

All of the attempts to genetically engineer plants with improved phytoremediation have previously been based on transformation of the nuclear genome. An alternative and novel approach is to engineer the chloroplast genomes of plants. This approach offers several advantages over nuclear transformation, i.e., very high levels of transgene expression (up to 46% of total protein (DeCosa et al., 2001), uniparental plastid gene inheritance (in most crop plants) that prevents pollen transmission of foreign DNA (Daniell et al., 1998; Daniell 2002, Daniell and Parkinson, 2003), the absence of gene silencing (Lee et al., 2003) and positioning effect (Daniell et al., 2001), the ability to express multiple genes in a single transformation event (DeCosa et al., 2001; Daniell and Dhingra, 2002), the ability to express bacterial genes without codon optimization (Kota et al., 1999; McBride et al., 1995; DeCosa et al., 2001), integration via a homologous recombination process that facilitates targeted transgene integration (Daniell et al., 2002) and sequestration of foreign proteins in the organelle which prevents adverse interactions with the cytoplasmic environment (Daniell et al., 2001; Lee et al., 2003). Engineering the chloroplast genome has successfully conferred insect resistance (McBride, et al., 1995; Kota et al., 1999; DeCosa et al., 2001), herbicide resistance (Daniell et al., 1998), disease resistance (De Gray et al., 2001), drought tolerance (Lee et al., 2003), expression of edible vaccines (Daniell et al., 2001), monoclonals (Daniell, 2003), and biopharmaceuticals (Fernandez-San Millan et al., 2003). Furthermore because the plastid genome of plants is highly conserved (Palmer, Trends in Genetics 6: 115-120, 1990; Sugiura, Plant Mol. Biol. 19: 149-168, 1992), the vectors described herein are likely to find applications in a large number of species.

Conservation of gene order in distantly related eubacteria appears to occur in operons that encode essential cellular apparatus, for example ribosomal and cell division proteins (Watanabe, H. et al. (1997). J. Mol. Evol. 44 (Suppl. 1), 57-64), or in operons in which the gene products physically interact. (Dandekar, T. et al. (1998) Trends Genet. 23, 324-328.

Indeed, the conservation of gene clusters coupled with the rapid increase in the availability of prokaryotic sequence data now allows the prediction of the functional coupling of genes and their assignment to metabolic pathways. (Overbeek, R. et al. (1999) The use of gene clusters to infer functional coupling. Proc. Natl. Acad. Sci. U.S.A. 96, 2896-2901).

Below is an exemplary list of suitable operons which can be inserted into the vectors described in the specification and used to phytoremediate contaminant compounds. It is well understood in the art that there is a myriad of such phytoremediating operons, which have been sequenced and characterized in terms of pathway, structure and function. As a result one skilled in the art will recognize that any identified and characterized phytoremediating operon is suitable for use in the invention.

Mercury (Hg), especially in organic form, is a highly toxic pollutant affecting plants, animals and man. In plants, the primary target of Hg damage is the chloroplast; Hg inhibits both electron transport and photosynthesis. In the present study, chloroplast genetic engineering is used for the first time to enhance the capacity of plants for phytoremediation. This was achieved by integrating a native operon containing the merA and merB genes (without any codon modification), which code for mercuric ion reductase (merA) and organomercurial lyase (merB), respectively, into the chloroplast genome in a single transformation event. Stable integration of the merAB operon into the chloroplast genome resulted in high levels of tolerance to the organomercurial compound, phenylmercuric acetate (PMA) when grown in soil containing up to 400 μM PMA: plant dry weights of the chloroplast transformed lines were significantly than those of wild type at 106, 200 and 400 μM PMA. That the merAB operon was stably integrated into the chloroplast genome was confirmed by PCR and Southern blot analyses. Northern blot analyses revealed stable transcripts, independently of the presence or absence of a 3′ untranslated region (3′ UTR) downstream of the coding sequence. The merAB dicistron was the more abundant transcript but also less abundant monocistrons were observed, showing that specific processing occurs between transgenes. The use of chloroplast transformation to enhance Hg phytoremediation is particularly beneficial because it prevents the escape of transgenes via pollen to related weeds or crops and there is no need for codon optimization to improve transgene expression. Chloroplast transformation may also have application to other metals that affect chloroplast function.

Still another exemplary operon constuct suitable for use in the present invention is the the ars operon of pR773, which is present in certain strains of E. coli, and consists of five genes: arsA, arsB, arsC, arsD and arsR, which are responsible for arsenic resistance. (Ji and Silver, 1995; Nies and Silver, 1995; Ramanathan, S., Ensor, M., Daunert, S. 1997. Bacterial biosensors for monitoring toxic metals. Trends in Biotechnology 15, 500-506. The plasmid-borne arsenical resistance (ars) operon encodes an arsenical-translocating ATPase and confers resistance to antimonials and arsenicals in Escherichia coli by extrusion of the toxic compounds from the cytosol. (J Biol Chem. 1993 Jan. 5;268(1):52-8).

Another suitable operon is the bph operon, which carries genes that encode for enzymes capable of the catabolism of pcbs. The bph operon is illustrated in FIG. 8. The bph operon is explained in J Biol Chem. 2000 Oct. 6;275(40):31016-23; J Bacteriol. 1993 August; 175(16):5224-32; and J Bacteriol. 2001 March; 183(5):1511-6. The bph operon has seven open reading frames consisting of bphA, E, F, G, B, C, and orf3 of unknown function. The large and small subunit (encoded by bphA and bphE), the ferredoxin (encoded by bphF), and the bphG encoded ferredoxin reductase comprise the enzyme called biphenyl dioxygenase which catalyzes the initial step in the aerobic degradation of PCBs

Still another operon suitable for use is the nah operon. The reactions involved in the bacterial metabolism of naphthalene to salicylate have been reinvestigated using recombinant bacteria carrying genes cloned from the NAH7 plasmid. When intact cells of Pseudomonas aeruginosa PAO1 carrying DNA fragments encoding the first three enzymes of the pathway were incubated with naphthalene, they formed products of the dioxygenase-catalyzed ring cleavage of 1,2-dihydroxynaphthalene. These products were separated by chromatography on Sephadex G-25 and identified by 1H and 13C nuclear magnetic resonance spectroscopy and gas chromatography-mass spectrometry as 2-hydroxychromene-2-carboxylate (HCCA) and trans-o-hydroxybenzylidenepyruvate (tHBPA). HCCA was detected as the first reaction product in these incubation mixtures by its characteristic UV spectrum, which slowly changed to a spectrum indicative of an equilibrium mixture of HCCA and tHBPA. Isomerization of either purified product occurred slowly and spontaneously to give an equilibrium mixture of essentially the same composition. tHBPA is also formed from HCCA by the action of an isomerase enzyme encoded by the NAH7 plasmid. The gene encoding this enzyme, nahD, was cloned on a 1.95 kb KpnI-BglII fragment. Extracts of Escherichia coli JM109 carrying this fragment catalyzed the rapid equilibrium of HCCA and tHBPA. Metabolism of tHBPA to salicylaldehyde by hydration and aldol cleavage is catalyzed by a single enzyme encoded by a 1 kb MluI-StuI restriction fragment. Mechanisms for both the isomerase- and hydratase/aldolase-catalyzed reactions are proposed. The salicylaldehyde dehydrogenase gene, nahF, was cloned on a 2.75 kb BamHI fragment which also carries the naphthalene dihydrodiol dehydrogenase gene, nahB. By identifying the enzymes encoded by various clones, the gene order for the nah operon was shown to be p, A, B, F, C, E, D. (Eaton, R. et al., 1994. Biotransformation of Benzothiophene by Isopropylbenzene-Degrading Bacteria. EPA/600/J-94/440. J. Bacteriol. 176(13):3992-4002. (ERL, GB 880). (Avail. from NTIS, Springfield, Va.: PB95-112199)

The NAH plasmid as described in (M. J. Simon, T. D. Osslund, R. Saunders, B. D. Ensley, S. Suggs, A. Harcourt, W. Suen, D. L. Cruden, T. Gibson, G. J. Zylstra, Gene, 127 (1993) 31-37.)). This operon has been described as being suitable for Bioremediation of Oil-Contaminated Soil.

Yet another another suitable operon, the cmt operon, is described in Eaton, R., 1997. J. Bacteriol. 179(10):3171-3180. (ERL, GB 993) and Eaton, R.,. 1996. J. Bacteriol. 178(5):1351-1362. (ERL, GB 941).

This references describes that Pseudomonas putida F1 utilizes p-cumate (p-isopropylbenzoate) as a growth substrate by means of an eight-step catabolic pathway. A 35.75-kb DNA segment, within which the cmt operon encoding the catabolism of p-cumate is located, was cloned as four separate overlapping restriction fragments and mapped with restriction endonucleases. By examining enzyme activities in recombinant bacteria carrying these fragments and sub-cloned fragments, genes encoding most of the enzymes of the p-cumate pathway were located. Subsequent sequence analysis of 11,260 bp gave precise locations of the 12 genes of the cmt operon. The first three genes, cmtAaAbAc, and the sixth gene, cmtAd, encode the components of p-cumate 2,3-dioxygenase (ferredoxin reductase, large subunit of the terminal dioxygenase, small subunit of the terminal dioxygenase, and ferredoxin, respectively); these genes are separated by cmtC, which encodes 2,3-dihydroxy-p-cumate 3,4-dioxygenase, and cmtB, coding for 2,3-dihydroxy-2,3-dihydro-p-cumate dehydrogenase. The ring cleavage product, 2-hydroxy-3-carboxy-6-oxo-7-methylocta-2,4-dienoate, is acted on by a decarboxylase encoded by the seventh gene, cmtD, which is followed by a large open reading frame, cmtI, of unknown function. The next four genes, cmtEFHG, encode 2-hy-droxy-6-oxo-7-methylocta-2,4-dienoate hydrolase, 2-hydroxypenta-2,4-dienoate hydratase, 4-hydroxy-2-oxovalerate aldolase, and acetaldehyde dehydrogenase, respectively, which transform the decarboxylation product to amphibolic intermediates. The deduced amino acid sequences of all the cmt gene products except CmtD and CmtI have a recognizable but low level of identity with amino acid sequences of enzymes catalyzing analogous reactions in other catabolic pathways. This identity is highest for the last two enzymes of the pathway (4-hydroxy-2-oxovalerate aldolase and acetaldehyde dehydrogenase [acylating]), which have identities of 66 to 77% with the corresponding enzymes from other aromatic meta-cleavage pathways. Recombinant bacteria carrying certain restriction fragments bordering the cmt operon were found to transform indole to indigo. This reaction, known to be catalyzed by toluene, 2,3-dioxygenase, led to the discovery that the tod operon, encoding the catabolism of toluene, is located 2.8 kb downstream from and in the same orientation as the cmt operon in P. putida F1.

Still another suitable operon, the ipb operon which has been shown to assist in the degradation of hydrocarbon pollution is described in Mulbry, W., 1991, Appl. Environ. Microbiol. 57(12):3679-3682. (ERL, GB X740). Specifically the paper identified a DNA segment involved in the regulation of the isopropylbenzene (cumene) catabolism operon (ipb) of plasmid pRE4 from Pseudomonas putida strain RE204 and Vibrio fischeri luciferase genes, luxCDABE, were used to create the ipbRo/pA′-luxCDABE reporter fusion plasmid, pOS25. Escherichia coli HMS174(pOS25) produces light in the presence of inducers of the ipb operon. These inducers were shown to be hydrophobic compounds including monoalkylbenzenes, substituted benzenes and toluenes, some alkanes and cycloalkanes, chlorinated solvents and naphthalenes. Complex hydrocarbon mixtures such as gasoline, diesel, and jet fuels (JP-4 and JP-5), and creosote, were also inducers of ipb-lux. Bacteria carrying the ibp-lux reporter may be useful as bioindicators of hydrocarbon pollution in the environment and may be particularly valuable for examining the bioavailability of inducing pollutants.

Still another exemplary operon is the dmp operon, which acts in the phenol degradative pathway. This operon and its related pathway are described in (American Society for Microbiology Appl Environ Microbiol. 2001 January; 67 (1): 162-171). Specifically, the reference shows that substrates and some structural analogues directly activate the regulatory protein DmpR to promote transcription of the dmp operon genes encoding the (methyl)phenol degradative pathway of Pseudomonas sp. strain CF600.

The Deinococcus radiodurans microbe, a particularly toxic resistant strain is believed to encode essential operons which are capable degrading highly toxic compounds and nuclear waste. (Nature., February 1999 Volume 17 Number 2 pp 137-138). The czc operon of Alcaligenes eutrophus CH34: provides a resistance mechanism to the removal of heavy metals. J Ind Microbiol 1995, 14:142-153.

Still another suitable and exemplary operon for use in the disclosed constructs is the cad operon. The cad operon is the resistance mechanism for cadmium was utilized in the construction of a cadmium specific sensor bacterium (Tauriainen, S., Karp, M., Chang, W., Virta, M. 1998. Luminescent bacterial sensor for cadmium and lead. Biosensors and Bioelectronics 13, 931-938.). The resistance for cadmium is encoded by the genes of the cadA operon, which consists of two genes: cadA and cadC (Yoon, K. P., Silver, S. 1991. A second gene in the Staphylococcus aureus cadA cadmium resistance determinant of plasmid pI258. J Bacteriol 173, 7636-7642.). The cadC gene encodes for the regulatory protein (Endo and Silver, 1995) and the cadA gene for an energy-dependent ion pump, which is responsible for efflux of cadmium from the cells (Tsai et al., 1992).

Another operon suitable for use in the current disclosure is the gene (todF) encoding 2-hydroxy-6-oxohepta-2,4-dienoate hydrolase. (Gene. 1991 Jul. 31;104(1):91-4). The todF gene in Pseudomonas putida F1 was shown to be located upstream of the todC1C2BADE genes. The latter form part of the tod operon and encode the enzymes responsible for the initial reactions in toluene degradation. The nucleotide (nt) sequence of todF was determined and the deduced amino acid (aa) sequence revealed that the hydrolase contains 276 aa with a Mr of 30,753. The deduced aa sequence was 63.5% homologous to that reported for 2-hydroxymuconic semialdehyde hydrolase which is involved in phenol degradation by Pseudomonas CF600. (Gene. 1991 Jul. 31;104(1):91-4).

These operons can be inserted into the vectors as described herein, or can be inserted by the methodology described in Applicants patent application Ser. No. 09/807, 723, which describes multiple gene expression in plant plastids. This application is fully incorporated by reference.

Of course this invention contemplates transforming plants without the use of operons, and instead transforming plastid genome with single genes not contained within an operon. An example of suitable phytoremediation genes include the genes involved in iron remediation. In all plants except grasses, iron acquisition which involves the reduction of highly insoluble ferric to the more soluble ferrous form, catalysed by the plasma membrane ferric reductase, prior to uptake by a Fe(D) transporter (Marschner 1995; Robinson et al. 1999).

The genes responsible for microbial metal resistance mechanism are organized in operons and are usually found in plasmids carried by the resistant bacteria (Ramanathan et al., 1997; Bruins et al., 2000). A number of exemplary genes genes suitable for bioremediation are described in Appl Microbiol Biotechnol (2001) 55:661-672.

The article published in Nature Biotechnology 19 (12): 1168-1172 December 2001, describes the phytodetoxification of TNT by transgenic plants expressing a bacterial nitroreductase. Specifically the article illustrates that there is major international concern over the wide-scale contamination of soil and associated ground water by persistent explosives residues. 2,4,6-trinitrotoluene (tnt) is one of the most recalcitrant and toxic of all the military explosives. the lack of affordable and effective cleanup technologies for explosives contamination requires the development of better processes. significant effort has recently been directed toward the use of plants to extract and detoxify tnt. to explore the possibility of overcoming the high phytotoxic effects of tnt, we expressed bacterial nitroreductase in tobacco plants. nitroreductase catalyzes the reduction of tnt to hydroxyaminodinitrotoluene (hadnt), which is subsequently reduced to aminodinitrotoluene derivatives (adnts). Transgenic plants expressing nitroreductase show a striking increase in ability to tolerate, take up, and detoxify tnt. our work suggests that expression of nitroreductase (nr) in plants suitable for phytoremediation could facilitate the effective cleanup of sites contaminated with high levels of explosives. (Nature Biotechnology 19 (12): 1168-1172 December 2001).

The publication In vitro cellular & developmental biology-plant 37 (3): 330-333 May-June 2001 describes the engineering of plants for the phytodetoxification of explosives. Specifically the reference describes the widespread contamination of the environment by explosives resulting from the manufacture, disposal and testing of munitions is becoming a matter of increasing concern. most explosives axe considered to he a major hazard to biological systems due to their toxic and mutagenic effects. Interest on the bioremediation of land contaminated with explosives has recently been focused on phytoremediation. unfortunately, whilst plants have many advantages for the remediation of contaminated land and water, they lack the catabolic versatility which enables microorganisms to mineralize such a wide diversity of xenobiotic compounds. This raised the interesting question as to whether the impressive biodegradative capabilities of soil bacteria could be combined with the high biomass and stability of plants to yield an optimal system for in situ bioremediation of explosive residues in soil. our investigation into the degradation of explosive residues by soil bacteria resulted in the isolation of enterobacter cloacae pb2, which is capable of utilizing nitrate ester explosives such as pentaerythritol tetranitrate (petn) and nitroglycerin as the sole source of nitrogen for growth. we have successfully introduced petn reductase, the enzyme initiating explosive degradation in this organism, into plants to create transgenic plants that degrade explosives. Since the bacterial degradative pathways for many classes of organic pollutant have been elucidated, this may be a generally applicable method of achieving bioremediation of contaminated soil in the environment.

The publication, Applied and Environmental Microbiology 68 (10): 4764-4771, October 2002, describes the cloning, sequencing, and characterization of the hexahydro-1,3,5-trinitro-1,3,5-triazine degradation gene cluster from Rhodococcus rhodochrous.

Hexahydro-1,3,5-trinitro-1,3,5-triazine (rdx) is a high explosive which presents an environmental hazard as a major land and groundwater contaminant. rhodococcus rhodochrous strain 11y was isolated from explosive contaminated land and is capable of degrading rdx when provided as the sole source of nitrogen for growth. Products of rdx degradation in resting-cell incubations were analyzed and found to include nitrite, formaldehyde, and formate. no ammonium was excreted into the medium, and no dead-end metabolites were observed. the gene responsible for the degradation of rdx in strain 11y is a constitutively expressed cytochrome p450-like gene, xpla, which is found in a gene cluster with an adrenodoxin reductase homologue, xplb. the cytochrome p450 also has a flavodoxin domain at the n terminus. This study is the first to present a gene which has been identified as being responsible for rdx biodegradation. the mechanism of action of xpla on rdx is thought to involve initial denitration followed by spontaneous ring cleavage and mineralization. (Applied and Environmental Microbiology 68 (10): 4764-4771, October 2002).

As described in Nature Biotechnology 17 (5): 491-494 May 1999 Plants offer many advantages over bacteria as agents for bioremediation; however, they typically lack the degradative capabilities of specially selected bacterial strains. Transgenic plants expressing microbial degradative enzymes could combine the advantages of both systems. To investigate this possibility in the context of bioremediation of explosive residues, we generated transgenic tobacco plants expressing pentaerythritol tetranitrate reductase, an enzyme derived from an explosive-degrading bacterium that enables degradation of nitrate ester and nitroaromatic explosives. Seeds from transgenic plants were able to germinate and grow in the presence of 1 mM glycerol trinitrate (GTN) or 0.05 mM trinitrotoluene, at concentrations that inhibited germination and growth of wild-type seeds. Transgenic seedlings grown in liquid medium with 1 mM GTN showed more rapid and complete denitration of GTN than wild-type seedlings. This example suggests that transgenic plants expressing microbial degradative genes may provide a generally applicable strategy for bioremediation of organic pollutants in soil. (Nature Biotechnology 17 (5): 491-494 May 1999).

Consequently, it was of interest to attempt to modify the plastid genome to engineer plants to have better phytoremediation capacity.

### SUMMARY OF THE INVENTION

One aspect of this invention provides for transformed plastid genomes engineered to enhance the capacity of plants for phytoremediation. Another aspect of this invention provides for at transformed plastid genome where a native bacterial operon is used for expression in plants without codon optimization. Still another aspect of this invention provides for plants and planst parts that are transformed to phytoremediate contaminant compounds. Still other aspects are include which provide for methods of phytoremediating polluted sites through the use of transformed plants, who have had their plastid genomes engineered to enhance the capacity of plants for the phytoremediation of contaminants.

Another aspect of this invention provides the modification of the plastid to make them capable of the removal and degradation of explosive and nitroaromatic contaminants from contaminated water in bioreactor systems. It was also desired to engineer plastids to sequester and detoxify heavy metals, common toxic copollutants of explosives.

### BRIEF DESCRIPTION OF THE DRAWINGS

FIG. 1 shows the bacterial bioassay of the mer operon.

FIG. 1A illustrates a Schematic representation of the transformed chloroplast genome: The map shows the transgenic chloroplast genome containing the pLDR-MerAB-3′UTR construct. The site of specific integration between trnI and trnA chloroplast genes is showed by the dotted line, specifying the homologous recombination sequences in the pLDR-MerAB-3′UTR and pLDR-MerAB. Landing sites for the 3P/3M and 5P/2M primer pairs used in PCR confirmation of integration, and expected sizes products are shown. BglII restriction digestion sites and the merAB probe used in the Southern blot analyses are shown. Fragments of 7.96 kb should be produced after the restriction digestion of the transgenic chloroplast genome respectively.

FIG. 1B shows the transformed E. coli grown in 100 μM HgCl₂. i). Transformed E. coli cells containing the vectors pLDR-MerAB and ii) pLDR-MerAB-3′UTR grown in LB at 100 μM HgCl₂. iii) Untransformed control (E. coli).

FIG. 1C, shows the effect of Mercuric chloride on E. coli Cell Proliferation. The transgenic clone pLDR-MerAB and pLDR-MerAB-3′UTR and the control E. coli were grown on liquid LB media with 25 μM and 50 μM of HgCl₂ for 24 hours at 37° C. The absorbance at 600 mm was measured.

FIG. 2, illustrates the PCR analysis of control and putative transformants.

FIG. 2A shows PCR products (1.65 kb) using 3P/3M primers show integration into the chloroplast genome. Lanes: 1: Marker; 2: pLDR-MerAB transgenic line; 3: pLDR-MerAB-3′UTR transgenic line; 4: Untransformed wild type.

FIG. 2B show the PCR products (3.8 kb) using 5P/2M primers confirms merAB integration. Lanes: 1: Marker; 2: pLDR-MerAB transgenic line; 3: pLDR-MerAB-3′UTR transgenic line; 4: Positive control (pLDR-MerAB plasmid DNA); 5: Untransformed wild type (Nicotiana tabacum var Petit Havana).

FIG. 3 shows the southern blot analysis using the flanking sequence probe and the merAB probe.

FIG. 3A shows the map demonstrating the wild type chloroplast genome, restriction digestion sites used for Southern blot analysis and the 0.81 kb flanking sequence probe.

FIG. 3B shows the transgenic lines (T0 generation) for the pLDR-MerAB lane 2 and the pLDR-MerAB-3′UTR lane 3, show the expected size fragment of 7.96 kb; The untransformed control, lane 1 shows the 4.47 kb fragment.

FIG. 3C shows lanes 1 and 2 which are positive T1 generation transgenic line, lane 3 is the untransformed control. In a and b, the flanking sequence probe was used.

FIG. 3D shows T0 transgenic lines, pLDR-MerAB lane 1 and pLDR-MerAB-3′UTR lane 2, and their respective T1 generation transgenic lines, lanes 4-5, show the 7.96 kb fragment. Untransformed wild type, lanes 3 and 6. The merAB probe was used inc.

FIG. 4 shows the northern blot analysis.

FIG. 4A illustrates the merA probe; transcripts of merAB dicistron (2,332 nt) and the merA monocistron (1,694 nt) are shown by arrows.

FIG. 4B shows the merB probe; transcripts for the merAB dicistron (2,332 nt), the aadA/merB dicistron (1,448 nt) and the merB monocistron (638 nt) are shown.

FIG. 4C shows the merAB probe; transcripts of the merAB dicistron (2,332 nt), the merA monocistron (1,694 nt), the aadA/merB dicistron (1,448 nt) and the merB monocistron (638 bp) are shown.

FIG. 4D shows the aadA probe; transcripts of the aadA/merB dicistron (1,448 nt) and the aadA monocistron (810 nt) are shown 0: Marker; 1: Wild Type, untransformed 2: pLDR-MerAB transgenic line; 3: pLDR-MerAB-3′UTR transgenic line.

FIG. 5 shows the relative effect of PMA concentration on the growth of wild type and transgenic lines of tobacco plants. Seeds were germinated in vitro on MS-medium (without sucrose and 0.5 g/mL spectinomycin). Seedling plants (10 days from germination) were transferred to a greenhouse and grown in soil for 6 days. Plants were then treated by adding 200 mL of 0, 50, 100, and 200 μM PMA supplied in Hoagland's nutrient solution. Photographs were taken 14 days after treatment. WT: negative control Petit Havana, 5A: pLDR-MerAB transgenic line, 9: pLDR-MerAB-3′UTR transgenic line.

FIG. 6 shows the effect of PMA on the total dry weight per plant of 24-day old wild type and transgenic tobacco plant lines grown on soil containing 0, 100, 200, 300 and 400 μM PMA for 14-d. WT: negative control Petit Havana, 5A: pLDR-MerAB transgenic line, 9: pLDR-MerAB-3′UTR transgenic line. Standard errors shown, n=5.

FIG. 7 shows the effect of PMA on total chlorophyll content (mg/g dry weight) of 15-mm diameter leaf disks excised from wild type and transgenic lines of tobacco and treated with 0 and 10 μM PMA for 6 days. WT: negative control Petit Havana, 5A: pLDR-MerAB transgenic line, 9: pLDR-MerAB-3′UTR transgenic line. Standard errors shown, n=5.

FIG. 8 shows a diagram for the bph operon of Pseudomonas sp. strain LB400, with specific enzymes labeled. The open ended reading frames are represented in the blue arrows, which indicate the direction of transcription. The operon has seven open reading frames consisting of bphA, E, F, G, B, C, and orf3 of unknown function. The large and small subunit (encoded by bphA and bphE), the ferredoxin (encoded by bphF), and the bphG encoded ferredoxin reductase comprise the enzyme called biphenyl dioxygenase which catalyzes the initial step in the aerobic degradation of PCBs.

FIG. 9 shows the gastight acrylic volatilization chambers used to collect the volatilized mercury from WT and chloroplast engineered pLDR-merAB and pLDR-merAB 3″UTR tobacco plants grown over a 13-day period on soil amended with 100 μM of either PMA or HgCl₂. Volatile Hg was quantitatively trapped in alkaline peroxide liquid traps solution (1:1 of 0.1% NaOH and 30% H₂O₂).

FIG. 10 shows the effects of mercury on the dry weight and root length of WT (black bars) and chloroplast engineered pLDR-merAB and pLDR-merAB 3″UTR (hatched bars) tobacco plants grown for 15 days on soil amended with PMA 100, 200, and 300 μM.

Values shown are the average±SE of 5 replicates. Significant difference (P<0.05) from the WT of the same treatment.

FIG. 11 shows the effects of mercury on the dry weight and root length of WT (black bars) and chloroplast engineered pLDR-merAB and pLDR-merAB 3″UTR (hatched bars) tobacco plants grown for 15 days on soil amended with 100, 200, and 300 μM HgCl₂. Values shown are the average±SE of 5 replicates. Significant difference (P<0.05) from the WT of the same treatment.

FIG. 12 shows the Hg concentration (μg/g dry weight) in shoots and roots of WT (black bars) and chloroplast engineered pLDR-merAB and pLDR-merAB 3″UTR (hatched bars) tobacco plants grown for 15 days on soil amended with 100, 200, and 300 μM of either PMA or HgCl₂.

Values shown are the average±SE of 5 replicates.

Significant difference at P<0.05 from the WT of the same treatment.

** Significant difference at P<0.001 from the WT of the same treatment

FIG. 13 shows the Hg L3 near-edge X-ray absorption spectra of PMA and HgCl₂ standards (A) and mer-AB transgenic tobacco shoots and roots treated with 100 μM of either PMA (13) or HgCl₂ (C).

FIG. 14 shows the X-ray absorption spectroscopy edge-fitting results for mer-AB transgenic tobacco plants roots treated with 100 μM PMA (A) or HgCl₂ (3). Values represent the percentage abundance of a particular chemical species of mercury.

FIG. 15 shows the rates of Hg[0] volatilization from WT (solid line) and chloroplast engineered pLDR-merAB (dotted line) and pLDR-merAB 3″UTR (dashed line) tobacco plants grown for 13 days on soil amended with 100 μM of either PMA (A) or HgCl₂ (B). Values shown are the average of 3 replicates.

Table 1 shows the t-values (unpaired t-test) comparing the differences in dry weight between each transgenic line of tobacco vs. wild type. 5A: pLDR-MerAB transgenic line; 9: pLDR-MerAB-3′UTR transgenic line.

TABLE 1

| | PMA concentration (μM) | | | | | | | | | |
|---|---|---|---|---|---|---|---|---|---|---|
| | Control | | 100 | | 200 | | 300 | | 400 | |
| | A | | A | | A | | A | | A | |
| Dry | .31 | .60* | .77* | .19** | .38** | .62** | .41 | .79 | .67** | .72** |
| weight | | | | | | | | | | |

*Significant at P < 0.05
**Significant at P < 0.001.

Table 2 shows the Hg accumulation in shoots and roots of WT and transgenic lines of tobacco plant grew in soil amended with 100, 200, and 300 μM of phenylmercuric acetate (PMA) or mercuric chloride (HgCl₂). WT: Wild type of Petit Havana, 5A: pLDR-MerAB transgenic line, 9: pLDR-MerAB-3′UTR transgenic line.

TABLE 2

| | PMA | | | | | | HgCl₂ | | | | | |
|---|---|---|---|---|---|---|---|---|---|---|---|---|
| | Shoot | | | Root | | | Shoot | | | Root | | |
| | WT | 5A | 9 | WT | 5A | 9 | WT | 5A | 9 | WT | 5A | 9 |
| 100 μM | 0.12 | 3.9** | 3.18 | 28.45 | 73.2** | 78.4** | 4.74 | 7.42 | 6.27 | 97.04 | 115.1 | 114.8 |
| 200 μM | 0.32 | 11.8* | 10.7* | 78.1 | 145.6* | 121.1** | 0.57 | 6.4** | 4.5* | 143.8 | 215.5 | 149.5 |
| 300 μM | 0.14 | 15.0** | 11.5** | 70.99 | 338.4** | 325.7** | 0.23 | 0.6* | 0.7* | 120.1 | 333.8* | 377.4 |

*Significant level at P < 0.05
**Significant level at P < 0.001

TABLE 3
Transgene Expression in chloroplasts

| Agronomic traits | Gene | Promoter | 5′/3′ Regulatory elements | Reference |
|---|---|---|---|---|
| Insect resistance | Cry1A(c) | Prrn | rbcL/Trps16 | Mc Bride et al |
| | | | | 1995 |
| Herbicide | CP4 (petunia) | Prrn | ggagg/TpsbA | Daniell et al |
| resistance | | | | 1998 |
| Insect resistance | Cry2Aa2 | Prrn | ggagg (native)/TpsbA | Kota et al 1999 |
| Herbicide | CP4 (bacterial or | Prrn | rbcL or T7 gene 10/Trps16 | Ye at al 2001 |
| resistance | synthetic) | | | |
| Insect resistance | Cry2Aa2 operon | Prrn | Native 5′UTRs/TpsbA | DeCosa et al |
| | | | | 2001 |
| Disease resistance | MSI-99 | Prrn | ggagg/TpsbA | DeGray et al |
| | | | | 2001 |
| Salt and drought | tps | Prrn | ggagg/TpsbA | Lee et al 2003 |
| tolerance | | | | |
| Phytoremediation | merA^{a}/merB^{b} | Prrn | ggagg^{a, b}/TpsbA | Ruiz et al 2003 |

| | | | 5′/3′ | | |
|---|---|---|---|---|---|
| Biopharmaceutical | | | regulatory | % tsp | |
| proteins | Gene | Promoter | elements | expression | Reference |
| Protein based polymer | EG121 | Prrn | T7gene10/ | Not tested | Guda et al |
| | | | TpsbA | | 2000 |
| Human somatotropin | hST | Prrn^{a}, | T7gene10^{a} or | 7.0%^{a} and | Staub et al |
| | | PpsbA^{b} | psbA^{b}/Trps16 | 1.0%^{b} | 2000 |
| Cholera toxin | ctxB | Prrn | ggagg/TpsbA | 4% | Daniell et al |
| | | | | | 2002 |
| Tetanus toxin | TetC | Prrn | T7 gene 10^{a}, | 25%^{a}, 10%^{b} | Tregoning et al |
| | (bacterial | | atpB^{b}/TrbcL | | 2003 |
| | and | | | | |
| | synthetic) | | | | |
| Human Serum Albumin | hsa | Prrn^{a}, | ggagg^{a}, psbA^{b}/ | 0.02%^{a}, | Fernandez-San |
| | | PpsbA^{b} | TpsbA | 11.1%^{b} | Milan et al |
| | | | | | 2003 |
| Interferon alpha 5 | INF□5 | Prrn | PpsbA/TpsbA | ND | Torres |
| Interferon alpha 2B | INF□2B | Prrn | PpsbA/TpsbA | 19% | Falconer |
| Interferon gamma | ifn-g | PpsbA | PpsbA/TpsbA | 6% | Leelavathi and |
| | | | | | Reddy, 2003 |
| Monoclonal antibodies | | Prrn | ggagg/TpsbA | ND | Daniell et al |
| | | | | | (photosynthesis) |
| Insulin like growth | Igf-1 | Prrn | PpsbA/TpsbA | 33% | Ruiz G |
| factor | | | | | |
| Anthrax protective | Pag | Prrn | PpsbA/TpsbA | 4-5% | Watson |
| antigen | | | | | |
| Plague vaccine | CaF1˜LcrV | Prrn | PpsbA/TpsbA | 4.6% | Singleton |

Table 3 lists as an illustrative example lists heterologous proteins expressed in chloroplasts and their regulatory sequences.

### DETAILED DESCRIPTION OF THE INVENTION

In one preferred embodiment, vectors are provided, which can be stably integrated into the plastid genome of plants for the expression of genes capable of phytoremediating contaminant compounds. In other preferred embodiments methods of transforming plastid genomes to express genes capable of phytoremediating contaminant compounds, transformed plants and progeny thereof, which express such genes are provided. Another aspect of this invention provides for a transformed plastid genome where a native bacterial operon is used for expression in plants without codon optimization. Still another aspect of this invention provides for plants and plant parts that are transformed to phytoremediate contaminant compounds. Still other aspects are include which provide for methods of phytoremediating polluted sites through the use of transformed plants, who have had their plastid genomes engineered to enhance the capacity of plants for the phytoremediation of contaminants.

The preferred embodiments of this application are applicable to all plastids of plants. These plastids include the chromoplasts, which are present in the fruits, vegetables, and flowers; amyloplasts which are present in tubers such as potato; proplastids in the roots of plants; leucoplasts and etioplasts, both of which are present in the non-green parts of plants.

Definitions

To better understand the current disclosure, the following definitions, which shall hold their meaning throughout this application unless otherwise noted, are provided to put the application in proper context.

As used herein, the terms “transformed”, “stably transformed” or “transgenic” with reference to a plant cell means the plant cell has a nucleic acid sequence integrated into its genome which is maintained through one or more generations.

The term “transgenic” means that the plant cell of the invention contains at least one foreign nucleic acid molecule(s) stably integrated in the genome.

As used herein, the term “plant part” includes any plant organ or tissue including, without limitation, seeds, embryos, meristematic regions, callus tissue, leaves, roots, shoots, gametophytes, sporophytes, pollen, and microspores. Plant cells can be obtained from any plant organ or tissue and cultures prepared therefrom. The class of plants which can be used in the methods of the present invention is generally as broad as the class of plants amenable to transformation techniques, including both monocotyledenous and dicotyledenous plants.

The term “regulatory regions or sequences”, which is well described in the art generally refers to a nucleic acid base sequence that aid in the control of gene expression.

An operon refers to any controllable unit of transcription consisting of structural genes transcribed together. An example of an operon is the oft cited lac operon. As reffered to herein, the term “phytoremediation operon” refers to any operon, which is capable of integrating into the plastid genome with the vectors and regulatory products described herein to transform a plant, and wherein the “phytoremediation operon” encodes for proteins, which when expressed in plant plastids make the transformed plant capable of facilitating the remedediation of contaminant compounds or contaminant or polluted water, and soil.y

Properly folded should be understood to mean a protein that is folded into its normal conformational configuration, which is consistent with how the protein folds as a naturally occurring protein expressed in its native host cell.

A contaminant is generally understood to be an impurity, or any material of an extraneous nature associated with a chemical, a pharmaceutical preparation, a physiologic principle, or an infectious agent.

A pollutant is generally understood to mean an undesired contaminant that results in pollution. Throughout this application interchangeable reference is made to the terms pollutant and contaminant.

Substantially homologous as used throughout the ensuing specification and claims, is meant a degree of homology to the native sequence in excess of 70%, most preferably in excess of 80%, and even more preferably in excess of 90%, 95% or 99%. Substantial sequence identity or substantial homology as used herein, is used to indicate that a nucleotide sequence or an amino acid sequence exhibits substantial structural or functional equivalence with another nucleotide or amino acid sequence. Any structural or functional differences between sequences having substantial sequence identity or substantial homology will be de minimis; that is, they will not affect the ability of the sequence to function as indicated in the desired application. Differences may be due to inherent variations in codon usage among different species, for example. Structural differences are considered de minimis if there is a significant amount of sequence overlap or similarity between two or more different sequences or if the different sequences exhibit similar physical characteristics even if the sequences differ in length or structure. Such characteristics include, for example, ability to maintain expression and properly fold into the proteins conformational native state, hybridize under defined conditions, or demonstrate a well defined immunological cross-reactivity, similar biopharmaceutical activity, etc. Each of these characteristics can readily be determined by the skilled practitioner in the art using known methods.

Spacer region is understood in the art to be the region between two genes. The chloroplast genome of plants contains spacer regions which highly conserved nuclear tide sequences. The highly conserved nature of the nuclear tide sequences of these spacer regions chloroplast genome makes the spacer region ideal for construction of vectors to transform chloroplast of a wide variety of plant species, without the necessity of constructing individual vectors for different plants or individual crop species. It is well understood in the art that the sequences flanking functional genes are well-known to be called “spacer regions”. The special features of the spacer region are clearly described in the Applicant's application Ser. No. 09/079,640 with a filing date of May 15, 1998 and entitled “Universal chloroplast integration of expression vectors, transformed plants and products thereof.” The aforementioned application Ser. No. 09/079,640 is hereby incorporated by reference. It was well-known that there are at least sixty transcriptionally-active spacer regions within the plant chloroplast genomes (Sugita, M., Sugiura M., Regulation of Gene Expression in Chloroplasts of Plants, Plant Mol. Biol., 32: 315-326, 1996). Specifically, Sugita et al. reported sixty transcriptionally-active spacer regions referred to as transcription units, as can be seen in Table II of the article. Because the transcriptionally active spacer regions are known, a universal vector, as described in the Applicant's U.S. patent application Ser. No. 09/079,640, can be used in the identified spacer regions contained within a variety of the plant chloroplast genomes. By utilizing the teachings in Sugita et al., intergenic spacer regions are easily located in the plastid genome. Consequently this allows one skilled in the art to use the methods taught in the Applicant's U.S. patent application Ser. No. 09/079,640 to insert a universal vector containing the psbA, the 5′ untranslated region (UTR) of psbA and the gene coding for HSA into the spacer regions identified by Sugita et al., and found across plants. The aforementioned applications and article are incorporated by reference.

Selectable marker provides a means of selecting the desired plant cells, vectors for plastid transformation typically contain a construct which provides for expression of a selectable marker gene. Of course as it is understood in the art a selectable marker is not required to transform the plastid genome. Rather the selectable marker provides a method to identify plants which have been transformed with the gene of interest. Marker genes are plant-expressible DNA sequences which express a polypeptide which resists a natural inhibition by, attenuates, or inactivates a selective substance, i.e., antibiotic, herbicide, or an aldehyde dehydrogenase such as Betaine aldehyde dehydrogenase (described in the Applicant's application Ser. No. 09/807,722 filed on Apr. 18, 2001, and herein fully incorporated by reference). Alternatively, a selectable marker gene may provide some other visibly reactive response, i.e., may cause a distinctive appearance or growth pattern relative to plants or plant cells not expressing the selectable marker gene in the presence of some substance, either as applied directly to the plant or plant cells or as present in the plant or plant cell growth media.

In either case, the plants or plant cells containing such selectable marker genes will have a distinctive phenotype for purposes of identification, i.e., they will be distinguishable from non-transformed cells. The characteristic phenotype allows the identification of cells, cell groups, tissues, organs, plant parts or whole plants containing the construct. Detection of the marker phenotype makes possible the selection of cells having a second gene to which the marker gene has been linked.

The use of such a marker for identification of plant cells containing a plastid construct has been described in the literature. In the examples provided below, a bacterial aadA gene is expressed as the marker. Expression of the aadA gene confers resistance to spectinomycin and streptomycin, and thus allows for the identification of plant cells expressing this marker. The aadA gene product allows for continued growth and greening of cells whose chloroplasts comprise the selectable marker gene product. Numerous additional promoter regions may also be used to drive expression of the selectable marker gene, including various plastid promoters and bacterial promoters which have been shown to function in plant plastids.

Inverted Repeat Regions are regions of homology, which are present in the inverted repeat regions of the plastid genome (known as IRA and IRB), two copies of the transgene are expected per transformed plastid. Where the regions of homology are present outside the inverted repeat regions of the plastid genome, one copy of the transgene is expected per transformed plastid.

Structural equivalent should be understood meaning a protein maintaining the conformational structure as the native protein expressed in its natural cell.

As is generally understood in the art the term “bioremediation” refers generally to The use of plants or microorganisms to clean up pollution or to solve other environmental problems. The use of the terms bioremediation and phytoremediation as used herein are interchangeable, and reference, without limitation, is made to both terms throughout the specification.

As is also generally understood in the art the term phytoremediation refers to the use of plants or algae to clean up polluted water or soil.

Pollution refers generally to the changing of a natural environment, either by natural or artificial means, so that the environment becomes harmful to the living things normally found in it. Most often this refers to the input of toxic chemicals into the environment.

Vectors

The current application contemplates the use of vectors capable of plastid transformation, particularly of chloroplast transformation. Such vectors include chloroplast expression vectors such as pUC, pBR322, pBLUESCRIPT, pGEM, and all others identified by Daniel in U.S. Pat. No. 5,693,507 and U.S. Pat. No. 5,932,479. Included are also vectors whose flanking sequences are located outside of the embroidered repeat of the chloroplast genome. These publications and patents are hereby incorporated by. Also incorporated by reference, is U.S. provisional application 60/393,651, and the Utility application filed based off of that provisonal.

One aspect of this invention utilizes the universal integration and expression vector competent for stably transforming the plastid genome of different plant species (universal vector). The universal vector is described in WO 99/10513 which was published on Mar. 4, 1999, and application Ser. No. 09/079,640 which was filed on May 15, 1998, wherein both of said references are incorporated in their entirety.

As an illustrative embodiment of the vectors, the Applicants created two vectors designated pLDR-MerAB-39-UTR and pLDR-MerAB, which were suitable for integration into the plastid genome of plants. Basic pLD vector, developed in this laboratory for chloroplast transformation, was used (Daniell et al., 1998; Daniell et al., 2001b; De Cosa et al., 2001; Guda et al., 2000; Kota et al., 1999). High levels of foreign protein expression in chloroplasts (3-21% of tsp) have been shown for different proteins using the SD 5′ sequence (Daniell et al., 2001b; DeGray et al., 2001; Kota et al., 1999).

However it should be noted that the two vectors are illustrative examples and vectors can be constructed with different promoters as was described in U.S. patent application Ser. No. 09/079,640, different selectable markers such as those described in U.S. patent application Ser. No. 09/807,722, and different flanking sequences suitable for integration into a variety of plant plastid genomes.

General Methodolgy for Transforming the Plastid Genome

This illustrative example shows generally all of the necessary steps to practice the Applicants invention. Of course other suitable methods, which are known in the art may be substittued or used to supplement the example methodolgy desribed herein.

Isolation of Genomic DNA from Plants.

Mortar and pestle, liquid nitrogen, fresh dark green leaves. DNeasy Plant Mini Kit (QIAGEN Inc.)

PCR Amplification of Chloroplast Flanking Sequence.

Materials for PCR reaction: Genomic DNA (50-100 ng/μl), dNTPs, 10× pfu buffer, Forward primer, Reverse primer, autoclaved distilled H₂O and Turbo pfu DNA Polymerase.

Vector Construction.

Plasmid pUC19 or pBlueScript SK (+/−).

Species specific PCR amplified chloroplast DNA flanking sequences.

A promoter functional in plastids, 5′UTR of chloroplast gene, selectable marker gene, gene of interest and chloroplast 3′UTR.

Restriction enzymes and buffers.

T4 DNA polymerase to remove 3′ overhangs to form blunt ends and fill-in of 5′ overhangs to form blunt ends or Klenow large fragment (fill-in of 5′ overhangs to form blunt ends), alkaline phoshatase for dephoshorylation of cohesive ends, DNA ligase to form phosphodiester bonds and appropriate buffers.

Water baths or incubators set at different temperatures.

Preparation for Biolistics.

Autoclaved Whatman filter paper #1 (55 mm in diameter) dried in oven.

100% ethanol.

Autoclaved tips in box, autoclaved kimwipes tissues wrapped in aluminum foil.

Sterile gold particles stored at −20° C. in 50% glycerol (see Notes 1 and 2).

Sterile rupture discs (1100 psi) and macrocarriers sterilized by dipping in 100% ethanol.

Autoclaved steel macrocarrier holders and stopping screens.

Freshly prepared 2.5 mM CaCl₂: weigh 1.84 g and dissolve in 5 mL H₂O and filter sterilized with 0.2 μm filter.

0.1 M spermidine (highly hygroscopic): dilute 1M spermidine stock to 10× and aliquot 100 μL in 1.5 mL Eppendrop tubes to store at −20° C. Discard each tube after single use.

Medium Preparation for Plant Tissue Culture.

2.5.1. Tobacco.

Medium for 1000 mL: 4.3 g MS salts (INVITROGEN Inc.), H₂O (molecular biology grade), 100 mg/L myo-inositol, 1 mg/L thiamine-HCl, 3% sucrose for shoot induction and 2% sucrose for root induction, 1 mg/L 6-benzyl aminopurine (BAP; use 1 mL from 1 mg/mL stock), 0.1 mg/L indole-3-acetic acid (use 0.1 mL from 1 mg/mL IAA stock), 1 mg/L indole-3-butyric acid for root induction (use 1 mL from 1 mg/mL IBA stock). Add 500 mg/L spectinomycin in autoclaved medium when it cools to 45° C.-50° C. (use 5 mL filter sterilized spectinomycin from 100 mg/mL stock).

Edible Crops.

Potato.

Medium for 1000 mL: 4.3 g MS salts, B5 vitamins (make 100× solution in 100 mL H₂O by dissolving: 1 g myo-inositol, 10 mg nictonic acid, 10 mg pyridoxine-HCl, 100 mg thiamine-HCl; use 10 mL, store remaining solution at 4° C.), 5 mg/l zeatin riboside (use 0.5 mL from 1 mg/mL ZR stock), 0.1 mg/l α-napthaleneacetic acid (use 0.1 mL from 1 mg/mL NAA stock), 40 to 500 mg/L spectinomycin.

Tomato.

Medium for 1000 mL: 4.3 g MS salts, B5 vitamins (10 mL from 10× stock), 0.2 mg/l indole-3-acetic acid (use 0.2 mL from 1 mg/mL IAA stock), 3 mg/l of 6-benzylaminopurine (use 3 mL from 1 mg/mL BAP stock). 300 or 500 mg/L spectinomycin.

For all plant growth media adjust to pH 5.8 with 1N KOH or 1N NaOH and add 6 g/L phytagel (Sigma) before autoclaving at 121° C. for 20 min. For preparation of 1 mg/mL stock of BAP, IAA, IBA, NAA, ZR respectively: weigh 10 mg powder and dissolve first in 1 or 2 drops of 1N NaOH and make up the final volume to 10 mL; store all plant growth regulators at 4° C. for 1-2 months).

Molecular Analysis of Transgenic Plants.

PCR Analysis for Gene Integration into Tobacco Chloroplasts

PCR reaction for 50 μL: 1.0 μl genomic DNA (50-100 ng/μl), 1.5 μl dNTPs (stock 10 mM), 5.0 μl (10× PCR buffer), 1.5 μl Forward primer (to land on the native chloroplast genome; stock 10 μM), 1.5 μl Reverse primer (to land on the transgene; stock 10 μM), 39.0 μl autoclaved distilled H₂O and 0.5 μl Taq DNA polymerase.

Analysis of Homoplasmy by Southern Blots

Depurination solution: 0.25 N HCl (use 0.4 mL HCl from 12.1 N HCl; Fisher Scientific USA, to make up final volume 500 mL with distilled H₂O).

Transfer buffer: 0.4 N NaOH, 1 M NaCl (weigh 16 g NaOH and 58.4 g NaCl and dissolve in distilled H₂O to make up the final volume to 1000 mL).

20×SSC: 3M NaCl, 0.3 M sodium citrate trisodium salt (weigh 175.3 g NaCl, 88.2 g Na₃C₆H₅O₇.2H₂O 900 mL H₂O and adjust pH 7.0 using 1 N HCl and make up the final volume to 1000 mL with distilled H₂O and autoclave).

2×SSC: Add 20 mL of 20×SSC in 180 mL of distilled H₂O.

Protein Analysis by Western Blots.

Acrylamide/Bis: ready made from Fischer (USA), stored at 4° C.

10% SDS: dissolve 10 g SDS in 90 mL deionized water, make up the volume to 100 mL, store at room temperature.

Resolving gel buffer: 1.5 M Tris-HCl (add 27.23 g Tris base in 80 mL water, adjust to pH 8.8 with 6 N HCl and make up the final volume to 150 mL. Store at 4° C. after autoclaving).

Stacking gel buffer: 0.5 M Tris-HCl (add 6.0 g Tris base in 60 mL water. Adjust to pH 6.8 with 6 N HCl. Make up the volume to 100 mL. Store at 4° C. after autoclaving).

Sample Buffer (SDS Reducing Buffer): In 3.55 mL water add 1.25 mL 0.5 M Tris-HCl (pH 6.8), 2.5 mL glycerol, 2.0 mL (10% SDS), 0.2 mL (0.5% Bromophenol blue). Store at room temperature. Add 50 μL β-Mercaptoethanol (βME) to 950 μL sample buffer prior to its use.

10× running buffer (pH 8.3): Dissolve 30.3 g Tris Base, 144.0 g Glycine and 10.0 g SDS in ˜700 mL water (add more water if not dissolving). Bring up the volume to 1 L and store at 4° C.

10× PBS: Weigh 80 g NaCl, 2 g KCl, 26.8 g Na₂HPO₄.7H₂O (or 14.4 g Na₂HPO₄), 2.4 g KH₂PO₄ in 800 mL water. Adjust pH to 7.4 with HCl and make up the volume to 1 L. Store at room temperature after autoclaving.

20% APS: Dissolve 200 mg ammonium persulfate in 1 mL water (make fresh every two weeks).

Transfer buffer for 1500 mL: Add 300 mL 10× running buffer, 300 mL methanol, 0.15 g SDS in 900 mL water and make volume to 1 L.

Plant Extraction Buffer:

| | | Used Concentration | Final Concentration |
|---|---|---|---|
| 60 | μl | 5M NaCl | 100 mM |
| 60 | μl | 0.5 M EDTA | 10 mM |
| 600 | μl | 1 M Tris-HCl | 200 mM |
| 2 | μl | Tween-20 | .05% |
| 30 | μL | 10% SDS | 0.1% |
| 3 | μL | BME | 14 mM |
| 1.2 | mL | 1 M Sucrose | 400 mM |
| 1 | mL | Water | |
| 60 | μL | 100 mM PMSF | 2 mM |

Add PMSF just before use (vortex to dissolve PMSF crystals).

PMSF (Phenylmethyl sulfonyl fluoride): Dissolve 17.4 mg of powdered PMSF in 1 mL of methanol by vortexing and store at −20° C. for up to a month.

Methods.

Isolation of Genomic DNA from Plants.

Extract the genomic DNA from fresh green leaves using DNeasy Plant kit (QIAGEN Inc.) following vender's instructions.

Amplification of Chloroplast Flanking Sequence.

Species-specific flanking sequences from the chloroplast DNA or genomic DNA of a particular plant species is amplified with the help of PCR using a set of primers that are designed using known and highly conserved sequence of the tobacco chloroplast genome.

Conditions for running PCR reaction: There are three major steps in a PCR, which are repeated for 30 to 40 cycles. (1) Denaturation at 94° C.: to separate double stranded chloroplast DNA. (2) Annealing at 54 to 64° C.: primers bind to single stranded DNA with formation of hydrogen bonds and the DNA polymerase starts copying the template. (3) Extension at 72° C.: DNA Polymerase at 72° C. extends to the template that strongly forms hydrogen bond with primers. Mismatched primers will not form strong hydrogen bonds and therefore, all these temperatures may vary based on DNA sequence homology. The bases complementary to the template are coupled to the primer on the 3′ side. The polymerase adds dNTPs from 5′ to 3′, reading the template in 3′ to 5′ direction and bases are added complementary to the template.

Chloroplast Transformation Vector.

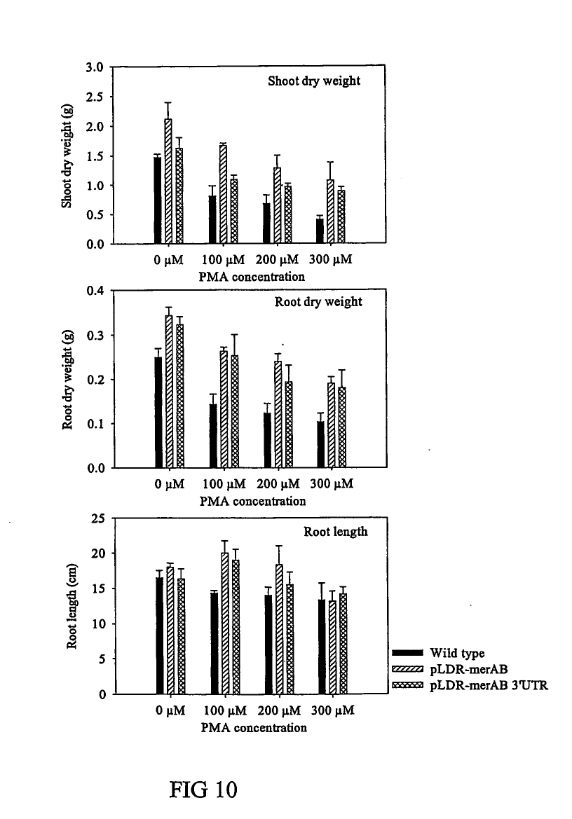

The left and right flanks are the regions in the chloroplast genome that serve as homologous recombination sites for stable integration of transgenes. A strong promoter and the 5′ UTR and 3′ UTR are necessary for efficient transcription and translation of the transgenes within chloroplasts. For multiple gene expression, a single promoter may regulate the transcription of the operon, and individual ribosome binding sites must be engineered upstream of each coding sequence (FIG. 10). The following steps are used in vector construction:

Amplification of flanking sequences of plastid with primers that are designed on the basis of known sequence of the tobacco chloroplast genome (between 16S-23S region of chloroplast).

Insert the PCR product containing the flanking sequence of the chloroplast genome into pUC19 plasmid digested with PvuII restriction enzyme (to eliminate the multiple cloning site), dephoshorylated with the help of alkaline phoshatase (CIP) for 5 min at 50° C. (to prevent recircularization of cloning vector). Inactivate CIP enzyme at 68° C. for 10 min.

Clone chloroplast transformation cassette (which is made blunt with the help of T4 DNA polymerase or Klenow filling) into a cloning vector digested at the unique PvuII site in the spacer region, which is conserved in all higher plants examined so far.

Delivery of Foreign Genes into Chloroplasts Via Particle Gun.

This is most successful and a simple technique to deliver transgenes into plastids and is referred as Biolistic PDS-1000/He Particle Delivery System. This technique has proven to be successful for delivery of foreign DNA to target tissues in a wide variety of plant species and integration of transgenes has been achieved in chloroplast genomes of tobacco, Arabidopsis, potato, tomato and transient expression in wheat, carrot, marigold and red pepper (see Note 5).

Preparation of Gold Particle Suspension.

Suspend 50-60 mg gold particles in 1 mL 100% ethanol and vortex for 2 min.

Spin at maximum speed ˜10,000×g (using tabletop microcentrifuge) for 3 min.

Discard the supernatant.

Add 1 ml fresh 70% ethanol and vortex for 1 min.

Incubate at room temperature for 15 min and shake intermittently.

Spin at 10,000×g for 2 min.

Discard supernatant, add 1 ml sterile distilled H₂O, vortex for 1 min, leave at room temperature for 1 min, and spin at 10,000×g for 2 min.

Repeat above washing process three times with H₂O (step 7).

Resuspend the gold-pellet in 1 mL 50% glycerol, store stock in −20° C. freezer.

Precipitation of the Chloroplast Vector on Gold Particles for Five Samples.

Take 50 μl the gold particles in 1.5 mL tube after vortexing for a 1 min.

Add 10 μl DNA (about 1 μg/μl plasmid DNA), and vortex the mixture for 30 sec.

Add 50 μl of 2.5 M CaCl₂ and vortex the mixture for 30 sec.

Add 20 μl of 0.1 M spermidine and vortex the mixture for 20 min at 4° C.

Washing of Chloroplast Vector Coated on Gold Particles.

Add 200 μl 100% ethanol and vortex for 30 sec.

Spin at 3000×g for 40 sec.

Pour off ethanol supernatant.

Repeat ethanol washings five times.

In the last step, pour off ethanol carefully and add 35-40 μl ethanol (100%).

Preparation of Macrocarriers.

Sterilize macrocarriers by dipping in 100% ethanol for 15 min and insert them into sterile steel ring holder with the help of a plastic cap when air-dried.

Vortex the gold-plasmid DNA suspension and pipet 8-10 μl in the center of macrocarrier and let it air dry.

Gene Gun Setup for Bombardment of Samples.

Wipe the gun chamber and holders with 1.00% ethanol using fine tissue paper (do not wipe the door with alcohol).

Turn on the vacuum pump.

Turn on the valve (Helium pressure regulator) of Helium gas tank (anti-clockwise).

Adjust the gauge valve (adjustable valve) ˜200 to 250 psi above the desired rupture disk pressure (clockwise) using adjustment handle.

Turn on the gene gun.

Place the rupture disc (sterilized by dipping in 100% ethanol for 5 min) in the rupture disc-retaining cap and tightly screw to the gas acceleration tube.

Place a stopping screen in the macrocarrier launch assembly and above that place macrocarrier with gold particles with chloroplast vector facing down towards screen. Screw assembly with a macrocarrier coverlid and insert in the gun chamber.

Place an intact leaf or explants to be bombarded on a filter paper (Whatman No. 1) soaked in medium containing no antibiotics. Place sample plate over target plate shelf, insert in the gun chamber and close the bombardment chamber door.

Press Vac switch to build pressure (up to 28 inches of Hg) in the vacuum gauge display. Turn same switch down at hold point and press Fire switch until you hear a burst sound of the ruptured disc.

Press Vent switch to release the vacuum and open the chamber to remove sample.

Shut down the system by closing the main valve (Helium pressure regulator) on the Helium gas cylinder. Create some vacuum in the gene gun chamber and keep using fire switch on and off until both pressure gauges' show zero reading. Release the vacuum pressure and turn off the gene gun and vacuum pump.

Incubate bombarded sample plates in the culture room for two days in the dark (i.e. covered with aluminum foil) and on the third day cut explants in appropriate pieces and place on the selection medium.

Plant Tissue Culture and Chloroplast transformation.

Tobacco Chloroplast Transformation.

A highly efficient and reproducible protocol has been established for Nicotiana tabacum cv. Petit Havana (Daniell, H. (1997) Methods in Mol. Biol. Recombinant gene expression protocols. 62,463-489.

Bombard 4 weeks old dark green tobacco leaves on the abaxial (bottom side) side with the chloroplast vector and incubate leaves in the dark for 2 days on selection free medium.

On the third day cut bombarded leaf explants into small square pieces (5 mm) and place explants facing abaxial surface towards selection medium containing MS salts, 1 mg/l thiamine HCl, 100 mg/l myo-inositol, 3% sucrose, 1 mg/l BAP and 0.1 mg/l IAA along with 500 mg/l spectinomycin as a selective agent.

Transgenic shoots should appear after three to five weeks of transformation. Cut the shoot leaves again into small square explants (2 mm) and subject to a second round of selection for achieving homoplasmy on fresh medium.

Regenerate transgenic shoots (confirmed by PCR for transgene integration) on rooting medium containing MS salts, 1 mg/l thiamine HCl, 100 mg/l myo-inositol, 2% sucrose and 1 mg/l IBA with 500 mg/l spectinomycin.

Transfer transgenic plants into pots under high humidity and move them to green house or growth chamber for further growth and characterization.

Plastid Transformation of Edible Crops.

The concept of universal vector for using the chloroplast DNA from one plant species to transform another species (of unknown sequence) was developed by the Daniell group. Using this concept both tomato and potato chloroplast genomes were transformed as described below.

Potato Chloroplast Transformation.

Using the tobacco chloroplast vector, leaf tissues of potato cultivar FL1607 was transformed via biolistics, and stable transgenic plants were recovered using the selective aadA gene marker and the visual green fluorescent protein (GFP) reporter gene.

Bombard potato leaves (3-4 week old) and incubate in the dark for 2 days on selection free medium.

Third day excise leaves into small square pieces (5 mm) and place on MS medium containing B5 vitamins, 5 mg/l ZR, 0.1 NAA, and 3% sucrose. Gradually increase spectinomycin selection pressure (40 to 400 mg/l) after every two weeks subculture under diffuse light.

Regenerate shoots from transgenic potato calli on MS medium containing B5 vitamins, 0.01 mg/L NAA, 0.1 mg/L GA3, 2% sucrose and 40-400 mg/l spectinomycin.

Transfer transgenic shoots on basal MS medium containing B5 vitamins, 2% sucrose and 40-400 mg/l spectinomycin for root induction. Transfer transgenic plantlets to growth chamber.

Tomato Chloroplast Transformation.

Using the tobacco chloroplast vector, tomato (Lycopersicon esculentum cv. IAC Santa Clara) plants with transgenic plastids were generated using very low intensity of light.

Bombard four-week-old tomato leaves and incubate in the dark for 2 days on selection free medium.

Excise bombarded leaves into small pieces and place on shoot induction medium containing 0.2 mg/L IAA, 3 mg/L BAP, 3% sucrose and 300 mg/L spectinomycin.

Select spectinomycin-resistant primary calli after a three to four month duration without any shoot induction.

Regenerate shoots in about four weeks after transfer of transgenic calli to shoot induction medium containing 0.2 mg/L IAA, 2 mg/L ZR, 2% sucrose and 300 mg/L spectinomycin then root on hormone-free medium. Transfer regenerated transgenic plants into the greenhouse.

Molecular Analysis of Transgenic Plants.

PCR Screening of Transgenic Shoots.

This method has been used to distinguish between mutants, nuclear and chloroplast transgenic plants. By landing one primer on the native chloroplast genome adjacent to the point of integration and a second primer on the aadA gene (PCR product of an appropriate size should be generated in chloroplast transformants. Since this PCR product cannot be obtained in nuclear transgenic plants or mutants, the possibility of nuclear integration or mutants should be eliminated.

Extract the genomic DNA from transgenic leaf tissue using DNeasy Plant kit (QIAGEN Inc.) by following vender's instructions. For lower amount of transgenic tissues, volume of buffers may be reduced appropriately.

Run PCR reaction with Taq DNA Polymerase (QIAGEN Inc.) using appropriate primers following the same conditions as described above for amplification of flanking sequences.

Analysis of Homoplasmy by Southern Blot.

In Southern blot analysis, tobacco plastid genome digested with suitable restriction enzymes should produce a smaller fragment (flanking region only) in wild type plants compared to transgenic chloroplast that include transgene cassette as well as the flanking region. In addition, homoplasmy in transgenic plants is achieved when only the transgenic fragment is observed.

Transfer of DNA to Membrane.

1. Digest the genomic DNA (˜2 to 10 μg) with suitable restriction enzymes from transgenic samples (including wild type as a control) and run digested DNA on 0.8% agarose gel containing 5 μL EtBr (from 10 mg/mL stock) in 100 mL for four hours at 40V.

2. Soak gel in 0.25 N HCl (depurination solution) for 15 minutes and rinse gel twice in distilled H₂O for 5 minutes.

3. Soak gel for 20 minutes in transfer buffer to denature DNA.

4. Transfer overnight DNA from gel to nylon membrane (pre-soak first in water, then in transfer buffer for 5 minutes) using the transfer buffer.

5. Next day, rinse membrane twice with 2×SSC buffer for 5 minutes each and air-dry for 5 minutes on filter papers. Cross-link transferred DNA to membrane using GS GeneLinker UV Chamber (Bio-Rad) at appropriate (C3) setting.

Preparation of Probe.

1. Digest any plasmid (containing flanking sequences of the chloroplast genome) with appropriate restriction enzymes.

2. Denature 45 μL flanking DNA fragment (50-250 ng) at 95° C. for 5 minutes, then place on ice for 2-3 minutes.

3. Add denatured probe to Ready-To-Go DNA Labeling Beads (-dCTP) tube (Amersham Biosciences, USA) and gently mix by flicking the tube.

4. Add 5 μL radioactive α³²P (dCTP; Amersham Biosciences, USA) to probe mixture and incubate at 37° C. for 1 hour and filter the probe using ProbeQuant G-50 Micro Columns (Amersham Pharmacia Biotech Inc. USA).

Prehybridization and Hybridization.

Place the blot (DNA transfer side facing towards the solution) in a hybridization bottle and add 10 mL Quik-Hyb (Stratagene, USA).

Incubate for 1 hour at 68° C. Add 100 μL sonicated salmon sperm (10 mg/mL stock; Stratagene, USA) to the labeled probe and heat at 94° C. for 5 minutes and add to bottle containing membrane and Quik-Hyb solution. Incubate for 1 hour at 68° C.

Washing and Autoradiography.

1. Discard Quik-Hyb solution with probe and wash membrane twice in 50 mL (2×SSC buffer and 0.1% SDS) for 15 minutes at room temperature.

2. Wash membrane twice in 50 mL (0.1×SSC buffer and 0.1% SDS) for 15 minutes at 60° C.

3. Wrap the wash membrane in saran wrap and expose blot to x-ray film in the dark and leave at −70° C. until ready for development.

Determination of Transgene Expression by Western Blot.

Extraction of Plant Protein.

1. Grind 100 mg of leaf in liquid nitrogen and add 200 μl of extraction buffer to samples on ice.

2. Add appropriate volume of freshly prepared 2× Sample loading buffer to an aliquot plant extract (from a stock containing 50 μL β-mercaptoethanol and 950 μL sample loading buffer).

3. Boil samples for 4 minutes with loading dye and centrifuge for 2 minutes at 10,000×g, then immediately load 20 μL samples into gel.

Running Gel.

Load samples on gel and run for half hour at 100 V, then 1 hour at 150 V until the marker bands corresponding to your protein are in middle.

Transfer of Protein to Membrane.

Transfer protein from gel to membrane using Mini Transfer Blot Module at 30 V overnight or 65 V for 2 hours or 100 V for 1 hour. Membrane wrapped in saran wrap can be stored at −20° C. for a few days if necessary.

Membrane Blocking.

1. After transfer, rinse membrane with water and incubate membrane in PTM (100 mL 1×PBS, 50 μL 0.05% Tween 20, and 3 g dry milk (3%) for 1 hour at room temperature.

2. Add primary antibody in suitable dilution for 15 mL and incubate for 2 hours at room temperature. Wash membrane twice with 1×PBS for 5 minutes each.

3. Add secondary antibody in proper dilution for 20 mL. Incubate for 1.5 hours at room temperature on a shaker.

4. Wash twice with PT (100 ml 1×PBS+50 μL Tween 20) for 15 minutes and finally with 1×PBS for 10 minutes.

Exposure of the Blot to X-Ray Film.

1. Mix 750 μL of each chemiluminescent solution (Luminol Enhancer and Stable Peroxide) in 1.5 mL tube and add to membrane, cover thoroughly.

2. Wipe out extra solution and expose blot to x-ray film for appropriate duration and develop film.

Seed Sterilization.

Vortex small amount of seeds into microcentrifuge tube with 1 mL 70% ethanol for 1 minute. Discard ethanol after brief spin.

Add 1 mL disinfecting solution (1.5% Bleach and 0.1% Tween 20) in tube and vortex intermittently for 15 min. Discard solution after brief spin.

Wash the seed thrice with sterile distilled water.

Spray seeds with sterile water on plate containing RMOP basal medium supplemented with 500 μg/mL spectinomycin to determine maternal inheritance in transgenic chloroplast plants.

Evaluation of Results.

Maternal Inheritance in Chloroplast Transgenic Plants.

Transgenes integrated into chloroplast genomes are inherited maternally. This is evident when transgenic seed of tobacco are germinated on RMOP basal medium containing 500 μg/mL spectinomycin. There should be no detrimental effect of the selection agent in transgenic seedlings whereas untransformed seedlings will be affected.

CTB-GM1-gangliosides Binding Elisa Assay.

Coat microtiter plate (96 well ELISA plate) with monosialoganglioside-GM1 {3.0 μg/mL in bicarbonate buffer (15 mM Na₂CO₃, 35 mM NaHCO₃, pH 9.6)} and as a control, coat BSA (3.0 ug/mL in bicarbonate buffer) in few wells.

Incubate plate overnight at 4° C.

Block wells with 1% (w/v) bovine serum albumin (BSA) in 0.01 M phosphate-buffered saline (PBS) for two hours at 37° C.

Wash wells thrice with PBST buffer (PBS containing 0.05% Tween 20).

Incubate plate by adding soluble protein from transformed and untransformed plants and bacterial CTB in PBS.

Add primary antibodies (rabbit anti cholera serum diluted 1:8000 in 0.01 M PBST containing 0.5% BSA) and incubate plate for 2 hours at 37° C.

Wash well thrice with PBST buffer.

Add secondary antibodies diluted 1:50,000 (mouse anti rabbit IgG-alkaline phosphatase conjugate in 0.01 M PBST containing 0.5% BSA) and incubate plate for 2 hours at 37° C.

Develop plate with Sigma Fast pNPP substrate. Stop reaction by adding 3 M NaOH and read plate absorbance at 405 nm.

The macrophage lysis assay is as follows:

Isolate crude extract protein from 100 mg transgenic leaf using 200 μL of extraction buffer containing CHAPS detergent (4% CHAPS, 10 mM EDTA, 100 mM NaCl, 200 mM Tris-HCI, pH 8.0, 400 mM sucrose, 14 mM β-mercaptoethanol, 2 mM PMSF) and one without CHAPS detergent.

Spin samples for five minutes at 10,000×g and use both supernatant and homogenate for assay

Plate macrophage cells RAW 264.7 (grown to 50% confluence) into 96-wells plate, incubated in 120 μL Dulbecco's Modified Eagle's Medium (DMEM; from Invitrogen life technologies).

Aspirate medium from wells and add 100 μL medium containing 250 ng/mL proteins in crude leaf extract.

In control plate, add only DMEM with no leaf fraction to test toxicity of plant material and buffers.

In another plate, add 40 μL dilutions onto RAW 264.7 cells from plant samples, which serially diluted 2 fold, so that the top row had plant extract at 1:14 dilution.

Add 20 μL of MTT 3-[4,5-dimethylthiazol-2-yl]-2,5-diphenyltetrazolium bromide; Sigma) to each well containing cells (from a stock 5 mg/ml MTT dissolved in 1×PBS and filter sterilize) after 5 hours to assess the cell death.

Incubate the plate at 37° C. for 5 hours. Remove media with needle and syringe. Add 200 μL of DMSO to each well and pipette up and down to dissolve crystals. Transfer to plate reader and measure absorbance at 550 nm.

Active PA was found in both the supernatant and homogenate fractions. However, maximum macrophage lysis activity was noticed in supernatant when extraction buffer was used with CHAPS detergent.

Cholera Toxin (CTB) Antigen as an Edible Vaccine.

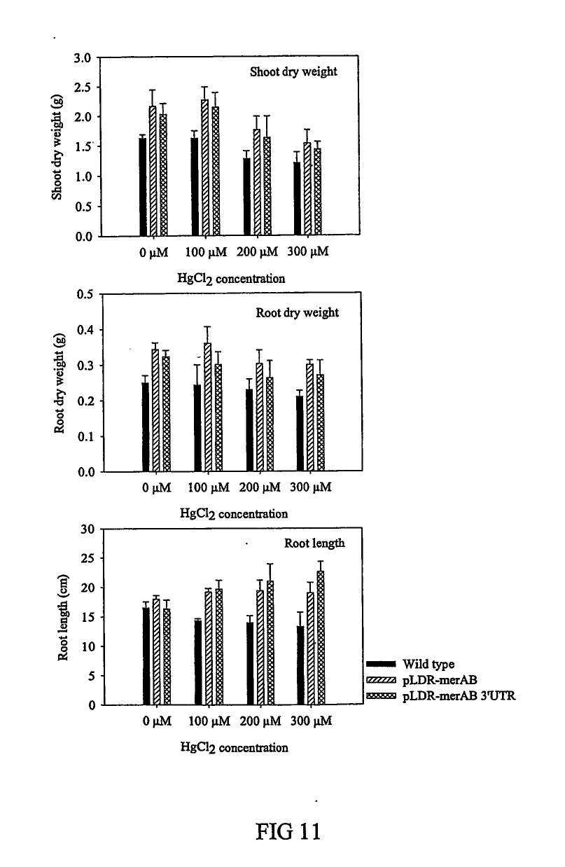

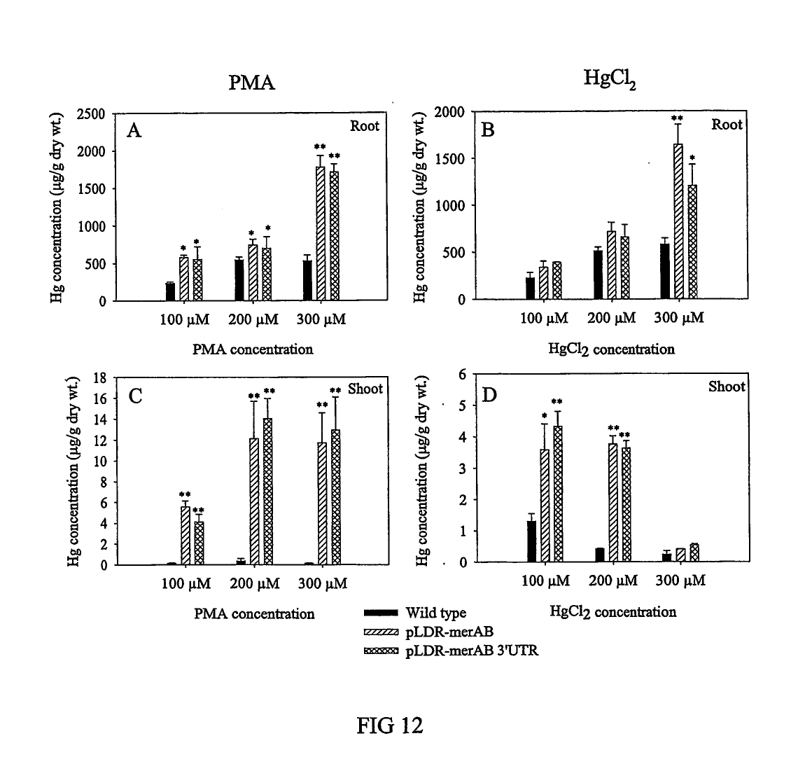

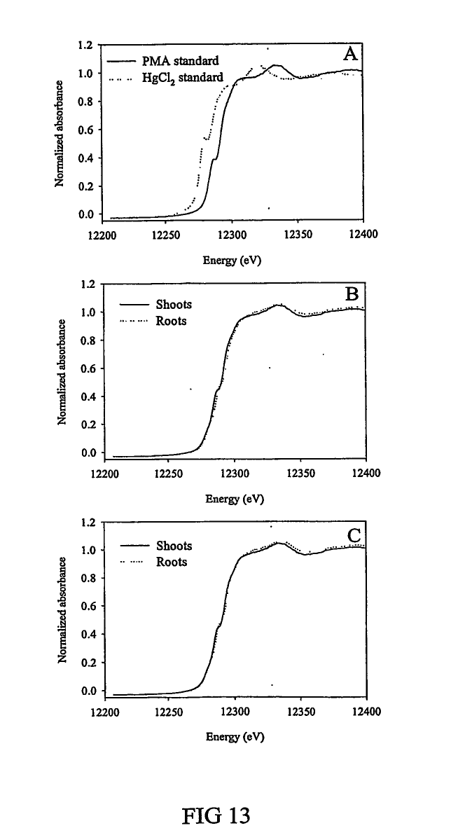

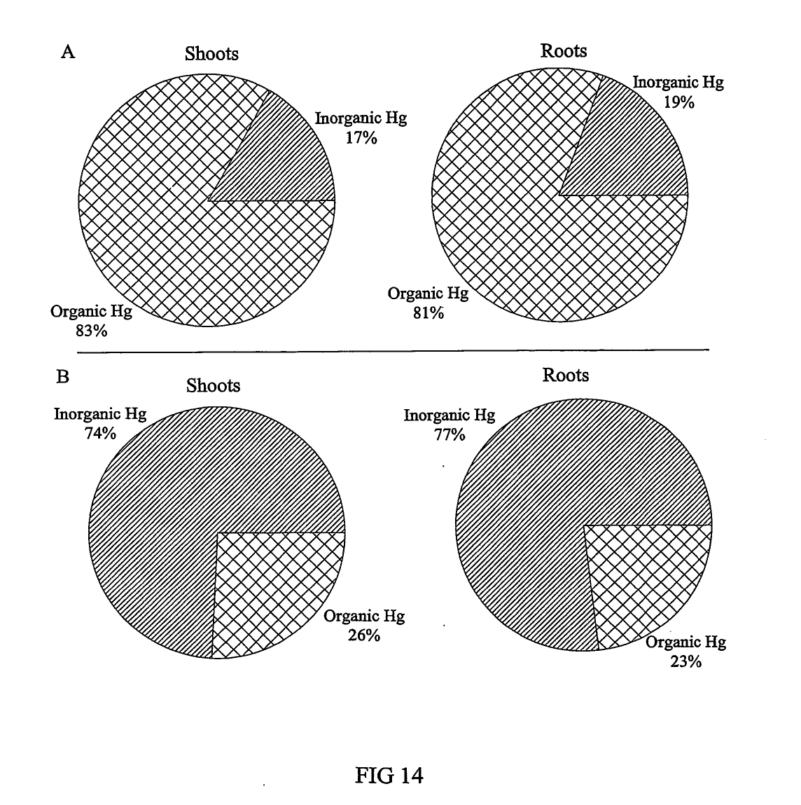

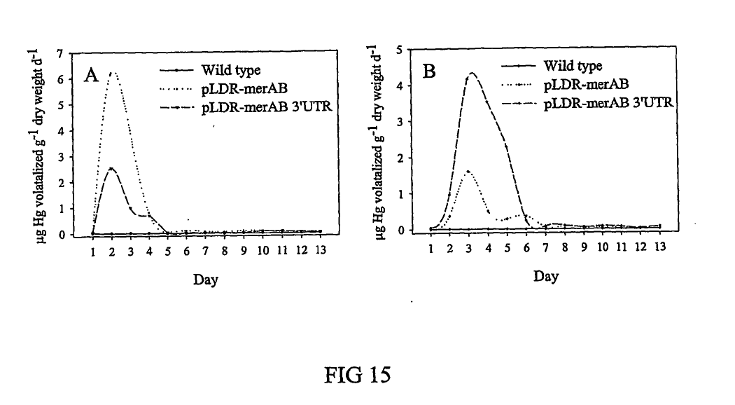

Chloroplast transgenic plants are ideal for production of vaccines. The heatlabile toxin B subunits of E. coli enterotoxin (LTB), or cholera toxin of Vibrio cholerae (CTB) have been considered as potential candidates for vaccine antigens. Integration of the unmodified native CTB gene into the chloroplast genome has demonstrated high levels of CTB accumulation in transgenic chloroplasts (Daniell, H., et al. (2001). J. Mol. Biol. 311, 1001-1009.). This new approach not only allowed the high level expression of native CTB gene but also enabled the multimeric proteins to be assembled properly in the chloroplast, which is essential because of the critical role of quaternary structure for the function of many vaccine antigens. The expression level of CTB in transgenic plants was between 3.5% and 4.1% tsp and the functionality of the protein was demonstrated by binding aggregates of assembled pentamers in plant extracts similar to purified bacterial antigen, and binding assays confirmed that both chloroplast-synthesized and bacterial CTB bind to the intestinal membrane GM1-ganglioside receptor, confirming correct folding and disulfide bond formation of CTB pentamers within transgenic chloroplasts (FIG. 11).

Oral Delivery of Vaccines and Selection of Transgenic Plants without the Use of Antibiotic Selectable Markers.

Betaine aldehyde dehydrogenase (BADH) gene from spinach has been used as a selectable marker to transform the chloroplast genome of tobacco (Daniell, H. et al., (2001) Curr. Genet. 39, 109-116). Transgenic plants were selected on media containing betaine aldehyde (BA). Transgenic chloroplasts carrying BADH activity convert toxic BA to the beneficial glycine betaine (GB). Tobacco leaves bombarded with a construct containing both aadA and BADH genes showed very dramatic differences in the efficiency of shoot regeneration. Transformation and regeneration was 25% more efficient with BA selection, and plant propagation was more rapid on BA in comparison to spectinomycin. Chloroplast transgenic plants showed 15 to 18 fold higher BADH activity at different developmental stages than untransformed controls. Expression of high BADH level and resultant accumulation of glycine betaine did not result in any pleiotropic effects and transgenic plants were morphologically normal and set seeds as untransformed control plants.

Production of Human Therapeutic Proteins in Transgenic Chloroplasts.

Human Serum Albumin (HSA) Protein.

Human Serum Albumin (HSA) accounts for 60% of the total protein in blood and widely used in a number of human therapies. Chloroplast transgenic plants were generated expressing HSA (Fernandez-San Millan et al., (2003) Plant Bitechnol. J. 1, 71-79). Levels of HSA expression in chloroplast transgenic plants was achieved up to 11.1% tsp. Formation of HSA inclusion bodies within transgenic chloroplasts was advantageous for purification of protein. Inclusion bodies were precipitated by centrifugation and separated easily from the majority of cellular proteins present in the soluble fraction with a single centrifugation step. Purification of inclusion bodies by centrifugation may eliminate the need for expensive affinity columns or chromatographic techniques.

Purification of HSA.

Solubilize the HSA inclusion bodies from transformed tissues using extraction buffer containing 0.2M NaCl, 25 mM Tris-HCl (pH 7.4), 2 mM PMSF and 0.1% Triton X-100.

Spin at 10,000×g. Suspend the pellet in buffer containing 6M Gu-HCl, 0.1M ME and 0.25 mM Tris-HCl (pH 7.4).

Dilute plant extract 100-fold in buffer containing 100 mM NaCl, 50 mM Tris-HCl (pH 8.5) and 1 mM EDTA.

Concentrate HSA protein by precipitation using a polyethylenglycol treatment at 37%.

Separate protein fractions by running a SDS-PAGE gel and stain gel with silver regent following vender's instruction (Bio-Rad, USA).

Electron Microscopy and Immunogold Labeling.

Cut the transformed and untransformed leaf in 1-3 mm squares.

Fix them in 0.1 M cacodylate buffer pH 7.4 (2.5% glutaraldehyde, 2% paraformaldehyde and 5 mM CaCl₂) for 15 minutes under vacuum and 12 hours at 4° C.

Rinse samples twice in 0.1M cacodylate buffer (pH 7.4) after fixation.

Dehydrate fixed samples through a graded ethanol series to 95%, then implant in LRW resin at 60° C. for 24 hours.

Cut ultra-thin sections using a Leica Ultracut T ultramicrotome and collect sections onto nickel grids.

Incubate sections in 0.05M glycine prepared in PBS buffer for 15 minutes to inactivate residual aldehyde groups.

Place grids onto drops of blocking solution (PBS containing 2% non-fat dry milk) and incubate for 30 minutes

Incubate sections for 1 hour in a goat anti-human albumin polyclonal antibody (dilution range from 1:1000 to 1:10,000 in blocking solution).

Wash sections with blocking solution 6×5 minutes each.

Incubate sections for 2 hours with a rabbit anti-goat IgG secondary antibody conjugate to 10 nm gold diluted 1:40 in blocking solution.

Wash sections 6×5 minutes in blocking solution and 3×5 minutes with PBS, and fixed sections in 2% glutaraldehyde diluted in PBS for 5 minutes.

Wash fixed sections in PBS 3×5 minutes, then in distilled water 5×2 min each.

Stain sections using uranyl acetate and lead citrate and examine samples under transmission electron microscope at 60 kv.

Notes.

Gold particles suspended in 50% glycerol may be stored for several months at −20° C. Avoid refreezing and thawing spermidine stock; use once after thawing and discard the remaining solution. Use freshly prepared CaCl₂ solution after filter sterilization. Do not autoclave.

Precipitation efficiency of DNA on gold and spreading of DNA-gold particles mixture on macrocarriers is very important. For high transformation efficiency via biolistics, a thick film of gold particles should appear on macrocarrier disks after alcohol evaporation. Scattered or poor gold precipitation reduces the transformation efficiency.

Generally, a 1000 bp flanking sequence region on each side of the expression cassette is adequate to facilitate stable integration of transgenes.

Use of the 5′ untranslated region (5′ UTR) and the 3′ untranslated region (3′ UTR) regulatory signals are necessary for higher levels of transgene expression in plastids (13). The expression of transgene in the plant chloroplast depends on a functional promoter, stable mRNA, efficient ribosomal binding sites; efficient translation is determined by the 5′ and 3′ untranslated regions (UTR). Chloroplast transformation elements Prrn, psbA5′UTR, 3′UTR can be amplified from tobacco chloroplast genome.

Bombarded leaves after two-days dark incubation should be excised in small square pieces (5-7 mm) for first round of selection and regenerated transgenic shoots should be excised into small square pieces (2-4 mm) for a second round of selection.

Temperature for plant growth chamber should be around 26-28° C. for appropriate growth of tobacco, potato and tomato tissue culture. Initial transgenic shoot induction in potato and tomato require diffuse light. However, higher intensity is not harmful for tobacco.

Transformation efficiency is very poor for both potato and tomato cultivars compared to tobacco.

Tobacco chloroplast vector gives low frequency of transformation if used for other plant species. For example, when petunia chloroplast flanking sequences were used to transform the tobacco chloroplast genome (DeGray, G. et al., (2001), Plant Physiol. 127,852-862.), it resulted in very low transformation efficiency.

Under diffuse light conditions, highly regenerating tomato cultivar (Microtom) shoots produce premature flowering that inhibit further growth of transgenic plants. Therefore, after the first shoot induction phase, shoots should be moved to normal light conditions.

### ILLUSTRATIVE EXAMPLE 1

Reference will now be made in detail to the presently preferred embodiments of the invention, which, together with the following example, serve to explain the principals of the invention. The following example is intended as a non-limiting example of the Applicants, and is no way to a intended as a limitation.

Mercury is a highly toxic element that is found both naturally or as an introduced contaminant in the environment (Patra and Sharma, 2000). It is usually found in the less toxic inorganic form which is extremely insoluble and chemically and physically stable (Gavis and Furgson 1972). Although the toxic effects of elemental mercury are low, the main problem is that elemental mercury can be converted to the highly toxic methylmercury through biological activities in soil and water (Nakamura et al., 1990, Meagher 2000), which may then be highly biomagnified up the food chain.

Traditional remediation strategies for mercury contaminated environments are expensive and site-destructive. On the other hand, phytoremediation, using plants to remediate contaminated environment, has been proposed as a cost-effective and environment friendly way for a large-scale clean up of contaminated sites (Doucleff and Terry 2002, Meagher and Rugh 1999).

Phenylmercuric acetate (PMA) was chosen to test the chloroplast transformation method because of the importance, toxicity of organomercurial compounds as environmental contaminants and because the site of action of organomercurial damage is the chloroplast (see above). The approach we used was to integrate a native operon containing the merA and merB genes, coding for mercuric ion reductase and organomercurial lyase, respectively, into tobacco chloroplast genomes. The results show that the chloroplast transgenic plants were substantially more resistant than wild type to the highly toxic organomercurial compound, PMA.

Results and Discussion

Chloroplast Vectors and Bacterial Resistance Assays

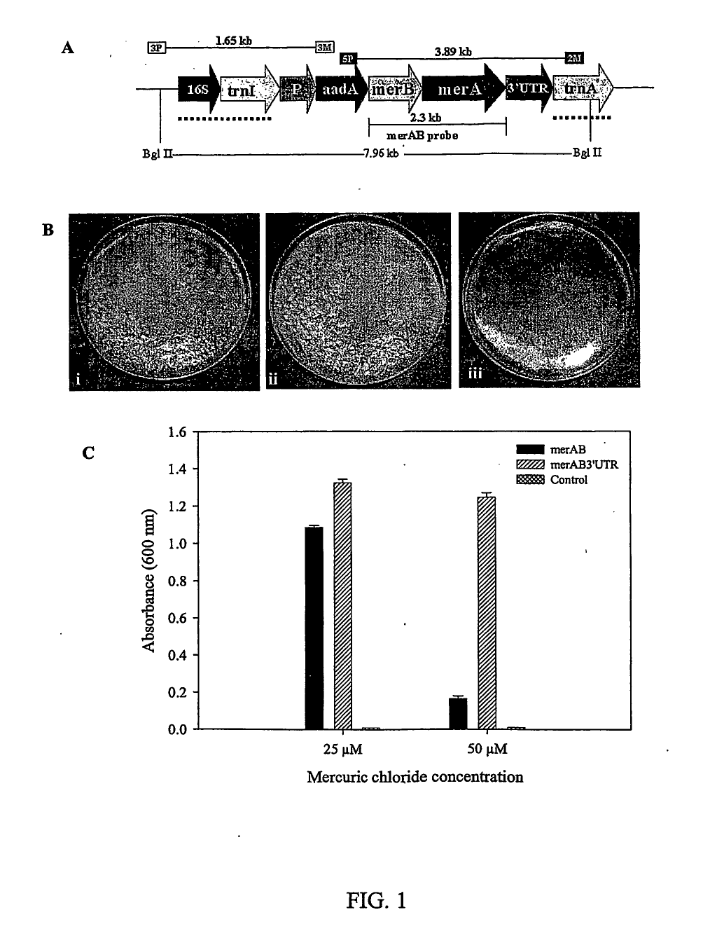

The bacterial native genes, merA (1.69 kb) and merB (638 bp) that encode the mercuric ion reductase and the organomercurial lyase, respectively, were amplified by PCR from E. coli strains harboring plasmids NR1 (containing the full length merA) and R831b (containing full the length merB). The PCR gene products were successively cloned into the pLD-vector which is a chloroplast specific vector used in previous publications from this laboratory (De Cosa et al., 2001; Daniell et al., 2001). This vector contains the homologous recombination sequences (flanking sequences) that allow site-specific integration of the operon containing the aadA, merB and merA genes into the inverted repeat region of the chloroplast genome in between the trnI (transfer RNA isoleucine) and trnA (transfer RNA alanine) genes (Daniell et al., 1998; Guda et al, 2000). The chloroplast 16S ribosomal RNA gene constitutive promoter (Prrn) drives the transcription of all downstream genes that include the aadA (aminoglycoside 3′-adenylyltransferase) gene conferring resistance to spectinomycin, the merA and merB genes. Two versions of the chloroplast vector were made with the presence or absence of the 3′untranslated region (UTR) from the chloroplast psbA gene that was expected to confer stability to transcripts, and they were designated pLDR-MerAB-3′UTR and pLDR-MerAB, respectively (FIG. 1A). The pLDR-MerAB-3′UTR and the pLDR-MerAB chloroplast vectors also contain the E. coli origin of replication and the ampicillin selectable marker that facilitates E. coli expression studies.

The transformed bacterial cells harboring pLDR-MerAB and pLDR-MerAB-3′UTR, and the control untransformed cells (E. coli) were grown on LB medium in the presence of different concentrations of mercuric chloride. Bacterial cells containing the pLDR-MerAB and pLDR-MerAB-3′UTR were able to grow in concentrations of HgCl₂ of up to 100 μM on solid agar plates (FIG. 1B). Untransformed E. coli cells were unable to grow even at a concentration of 25 μM (FIG. 1B). While transformed cells were able to grow in liquid broth at concentrations of 25 and 50 μM HgCl₂, differences in the rate of growth between the clone transformed with the plasmid containing the 3′ terminator and the clone that lacked the terminator region were examined (FIG. 1C). It is known from previous studies that the 3′ UTR's in E. coli are engaged in the termination of transcription. The pLDR-MerAB-3′UTR was expected to grow better in the presence of Hg because, by terminating effectively, more copies of a shorter transcript containing the merAB operon would be made, in contrast to fewer longer transcripts in the case of the pLDR-MerAB clone. The Hg bioassay showed that indeed E. coli cells transformed with the pLDR-MerAB-3′UTR vector resulted in bacterial growth when compared with the bacterial cells containing the vector lacking a 3′ psbA untranslated region (FIG. 1C).

Transformation, Selection and Characterization of Chloroplast Transgenic Plants

Chloroplast transgenic plants were obtained as described (Daniell, 1997). More than 20 positive independent transgenic lines were obtained with each construct. In this report, we show the results of two transgenic lines that were transformed with the pLDR-MerAB vector and the pLDR-MerAB-3′UTR, respectively. The variability in expression levels among independent chloroplast transgenic lines were minimal as reported previously (Daniell et al., 2001) and the results shown here correlate well with the results of other transgenic lines with the same chloroplast vectors.

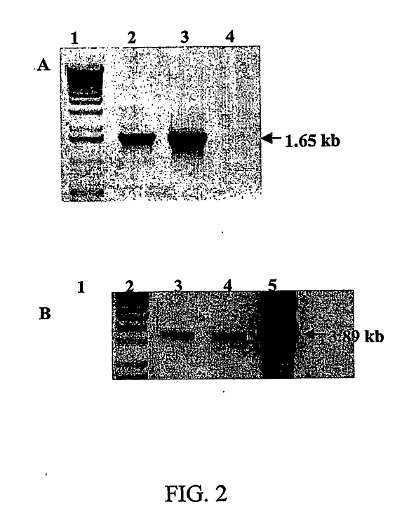

The primer pair 3P and 3M was used to test integration of the transgene cassette into the chloroplast genome at very early stages during the selection process. The 3P primer lands in the native chloroplast genome and the 3M primer lands in the aadA gene that is present within the gene cassette (FIG. 1A). If integration has occurred, a 1.65 kb PCR product should be obtained (FIG. 2A). The untransformed control and the mutants (caused by the spontaneous mutation of the 16S rRNA gene which confers resistance to spectinomycin) did not show any product, confirming that these plants are negative for integration of transgenes (FIG. 2A). The integration of transgenes (aadA, merA and merB), was further tested by using the 5P/2M primers and PCR analysis. The 5P and 2M primers annealed to the internal region of the aadA and trnA genes, respectively (FIG. 1A). The product size of positive transgenic clones was 3.89 kb, while the mutants and untransformed control did not show any PCR product (FIG. 2B).

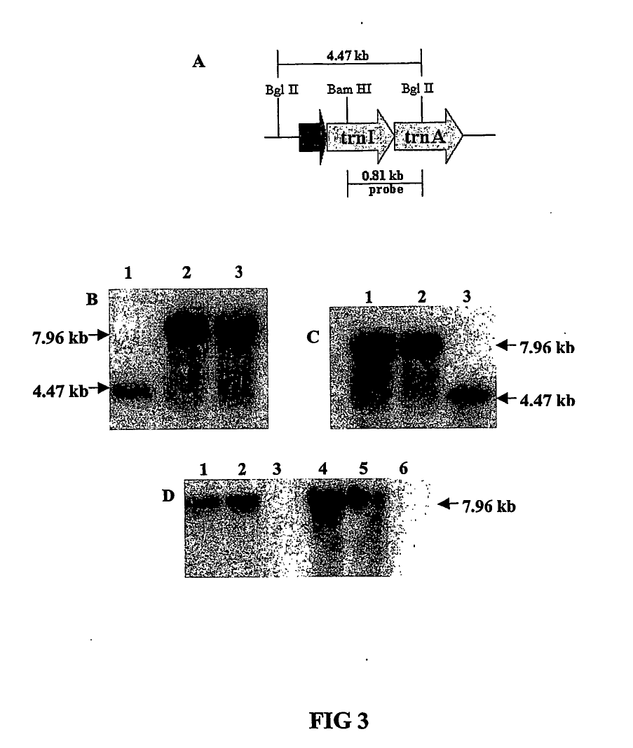

The DNA from full-grown T0 and T1 generation plants was extracted and used for the Southern blot analysis (FIG. 3). The 0.81 kb flanking sequence probe that hybridizes with the trnI and trnA genes (FIG. 3A) allowed detection of the site-specific integration of the gene cassette into the chloroplast genome. The transformed chloroplast genome digested with BglII restriction enzyme produced a fragment of 7.96 kb (FIG. 1A, 3B, 3C). The untransformed chloroplast genome digested with BglII yielded a 4.47 kb fragment (FIG. 3A, 3B, 3C). The flanking sequence probe also showed that homoplasmy of the chloroplast genomes was achieved through the selection process. Southern blots confirmed stable integration of foreign genes into all of the chloroplast genomes confirming homoplasmy. T0 and T1 generation transgenic plants only showed a single fragment of 7.96 kb. Absence of any detectable native untransformed chloroplast genomes not only confirmed homoplasmy but also facilitated detection of transgene copy numbers in each cell. It is known that mature leaf cells in tobacco contain about 10,000 copies of chloroplast genomes per cell (Bendich, 1987). By virtue of achieving homoplasmy, it is inferred that there are 10,000 copies of transgenes per cell. Southern blots detected with the merAB probe (2.3 kb in size) showed integration of specific genes, merA and merB as a single fragment of 7.96 kb (FIG. 1A, 3D). The control untransformed tobacco plants and mutants did not show this fragment (FIG. 3D). If the merAB probe would have detected any unexpected size fragments, it might be a non-specific integration into other plant genomes (nuclear or mitochondria) as discussed elsewhere (Daniell and Parkinson, 2003); but this was not observed. The transgenic plants were fully characterized via PCR and Southern Blot analysis, which showed site-specific integration of the genes into the chloroplast genome and achievement of homoplasmy even at very early stages of selection (T0). No difference in homoplasmy was detected among plants transformed with the pLDR-MerAB or pLDR-MerAB-3′UTR vector.

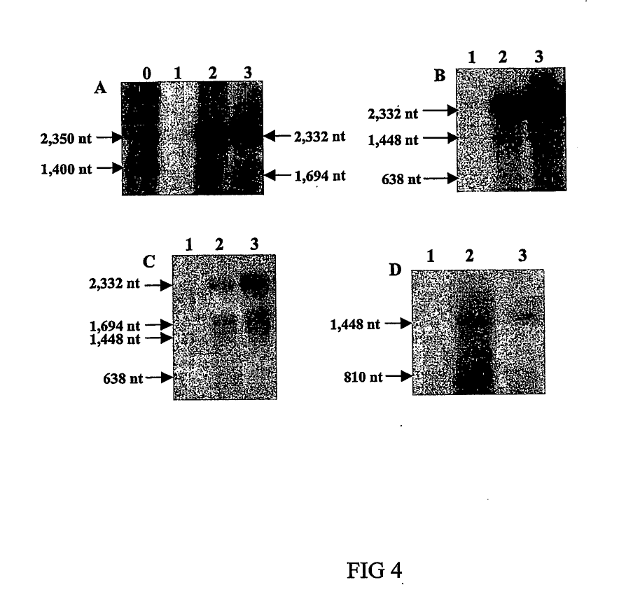

Total RNA from T0 and T1 plants transformed with the pLDR-MerAB-3′UTR and the pLDR-MerAB was extracted and used to perform the northern blot analysis with four different probes (the merA, merB, merAB and aadA probes). The merA probe clearly showed the dicistron containing the merB and merA genes with sizes of 2,332 nt and also a minor transcript for the merA monocistron of 1,694 nt (FIG. 4A). The merB probe showed the merAB dicistron (2,332 nt) plus a less abundant transcript (1,448 nt) containing the aadA and merB genes, and the monocistron corresponding to the merB (638 nt) transcript (FIG. 4B). The merAB probe helped to visualize different transcripts in a single blot, the merB and merA dicistronic transcript (2,332 nt), the merA monocistron (1.694 nt), the aadA and merB dicistron (1,448 nt) and the merB monocistron (638 nt) (FIG. 4C). The aadA probe showed transcripts for the dicistron containing the aadA and merB genes and also the aadA monocistron of 810 nt (FIG. 4D). The northern blot analyses showed that the most abundant transcript is the dicistron (2,332 nt) containing the merA and merB genes. Less abundant transcripts corresponding to the aadA/merB dicistron (1,448 nt), the merA monicistron (1,694 nt), the merB monocistron (638 nt) and to the aadA monocistron (810 nt) were also detected. The high abundance of the merAB dicistron in either the pLDR-MerAB or the pLDR-MerAB-3′UTR plants is an interesting observation. Contrary to the current dogma in the literature, these transcripts were stable even in the absence of a 3′ UTR, believed to be required for transcript stability. In addition, there is indication that processing occurs in between transgenes in transgenic chloroplasts even though no such processing sequences were engineered. Even though all three transgenes genes are transcribed from a single promoter, no tricistrons containing the aadA, merB and merA genes were detected. Observed processing between transgenes might be due to recognition of bacterial intergenic sequences by the chloroplast protein synthesis machinery.

Bioassays

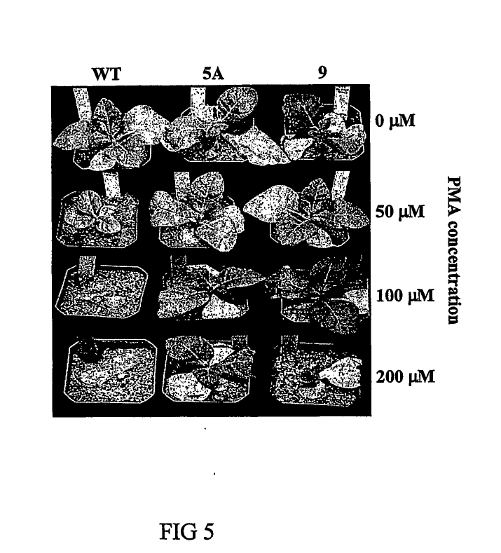

When 16-day-old tobacco plants were grown for 14 days in soil containing PMA concentrations of 0, 50, 100 and 200 μM, the merAB seedlings (both pLDR-MerAB and pLDR-MerAB-3′UTR clones) grew well at PMA concentrations up to 100 μM PMA, and survived the highest PMA concentration of 200 μM (FIG. 5). On the other hand, PMA concentrations of 100 and 200 μM PMA were lethal to wild type plants, which barely survived 50 μM PMA (FIG. 5). There were no significant differences between transgenic lines with or without the 3′ UTR terminator.

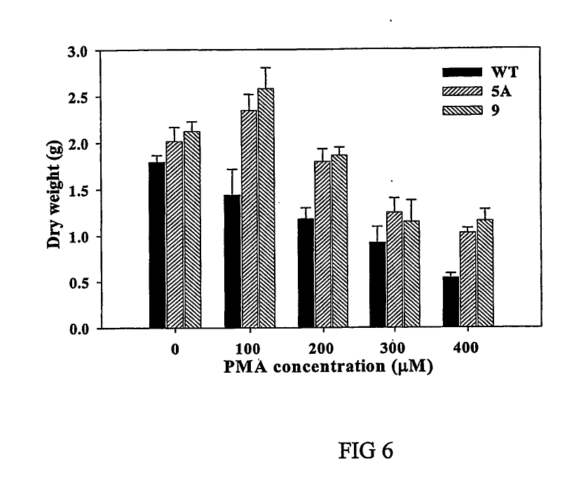

The effect of PMA on plant growth was determined by treating 24-day-old tobacco plants with PMA concentrations of 0, 100, 200, 300 and 400 μM in soil and measuring total plant dry weight at each concentration (FIG. 6). The total dry weight of wild type plants decreased progressively with each increase in PMA from 0 to 400 μM. In the transgenic plants on the other hand, there was no decrease in total dry weight with increase in PMA concentration until PMA reached 400 μM. Statistical analysis (unpaired t-test) showed that the transgenic lines were substantially more resistant than wild type to concentrations of PMA of 100, 200 and 400 μM (Table 1). These results indicate clearly that, compared to the wild type, the insertion of merA and merB into the chloroplast genome substantially increased the resistance of the transgenic plants to the toxic effects of PMA. There was no significant difference between the dry weights of the two clones, pLDR-Mer and pLDR-Mer-3′UTR, at each concentration of PMA tested (FIG. 6).

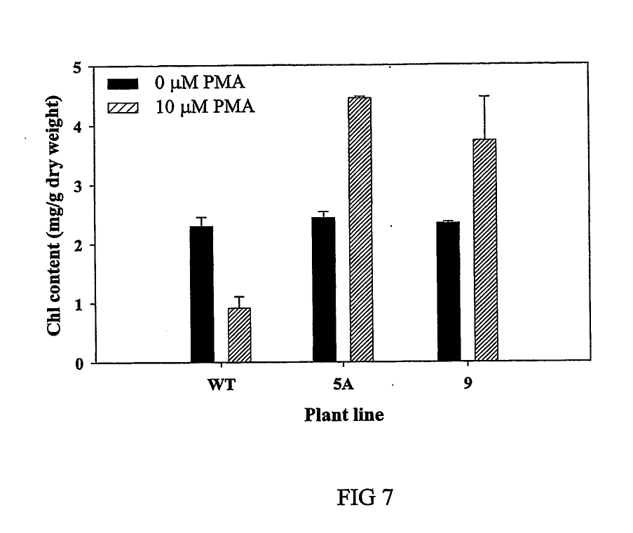

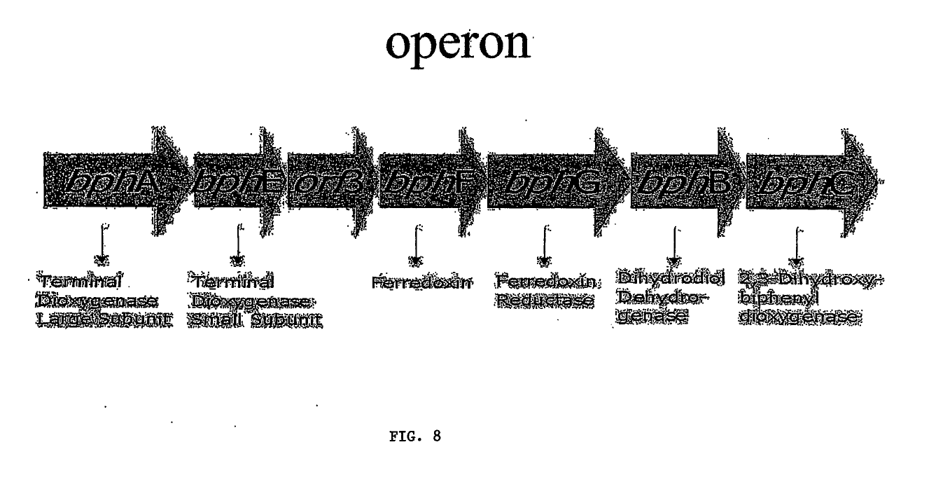

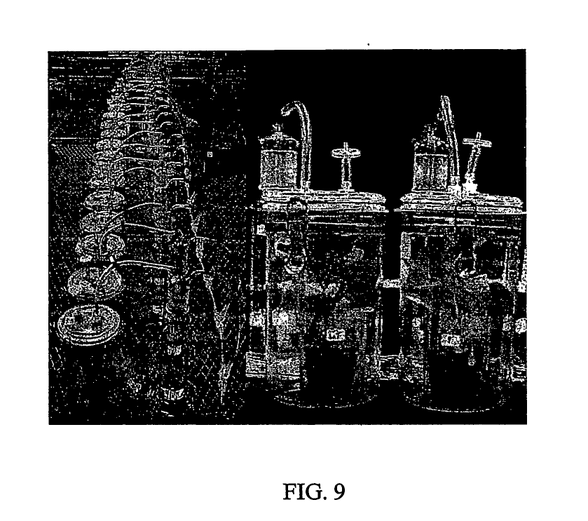

As discussed in the Introduction, previous research has shown that the main site of damage of organomercurial compounds is the chloroplast, and that chlorophyll synthesis, electron transport and photosynthesis are all seriously affected. The overexpression of merA and merB in the chloroplast should therefore reduce the toxic effects of PMA directly on chloroplast function. To test this idea, we treated 15-mm diameter leaf disks excised from wild type and transgenic plants with 10 μM PMA for 10 days and measured chlorophyll contents (FIG. 7). The results show that without PMA present chlorophyll concentration did not differ between wild type and the two transgenic lines. Surprisingly, when PMA was supplied to the leaf disks, the chlorophyll content was markedly increased in the transgenic lines, while in the wild type, chlorophyll content was reduced. These results are consistent with the view that PMA exerts a damaging effect on the chloroplasts of wild type plants as expected, reducing chlorophyll content substantially, and that overexpression of merA and merB in the chloroplast genome appears to increase chloroplast resistance to PMA toxicity. However, since the overexpression of these genes results in an increase in chlorophyll content of the transgenic chloroplasts, it would appear that PMA could in fact stimulate chlorophyll synthesis in some way in these transgenic plants. In this regard, it is of interest that the leaf disks taken from the transgenic plants increased in size over the 10-day experimental period, whereas disks from the wild type decreased in size. Thus, it is possible that the increase in chlorophyll concentration with PMA in the transgenic plants was associated with an increase in chloroplast number and/or size.

Levels of transgene expression in chloroplasts could be further enhanced by introducing appropriate UTRs instead of the RBS used in the present study. For example, we have recently shown that use of the psbA 5′ UTR instead of RBS resulted in a 500-fold increase in the expression of Human Serum Albumin in transgenic chloroplasts (Fernandez-San Millan et al., 2003). The most significant advantage is the ability to introduce the mer operon in a single transformation event in contrast to nuclear transgenic plants that required introduction of single genes followed by time consuming back crosses to reconstitute the entire pathway. In addition, prokaryotic genes do not require codon optimization when expressed in transgenic chloroplasts (Kota et al., 1999; DeCosa et al., 2001).

This example provides the first report on the use of chloroplast transformation using multigene engineering for the phytoremediation of toxic compounds. Because of the containment of transgenes and high levels of expression via chloroplast genomes, the chloroplast transformation approach is highly suitable for phytoremediation, especially for toxic agents that affect chloroplast function. Although 3′ UTR is believed to stabilize chloroplast transcripts and essential for transgene expression, it may not be necessary for transcript stability, in the context of a polycistron. Because there are more than sixty such polycistrons within the chloroplast genome (Sugita and Sugiura, 1996), this is a significant observation.

Experimental Potocol

Host E. coli cells containing plasmids NR1 and R831b were kindly provided by Dr. Ann Summers, University of Georgia. These plasmids contain the mer operon with the complete and functional merA and merB genes, respectively (Jackson and Summers 1982; Rinderle et al., 1983; Ogawa et al., 1984; Begley et al., 1986). Each of these plasmids confers resistance to at least one antibiotic that can be used as a selectable marker. Host bacterial containing plasmid NR1 was grown on solid LB media containing 100 μg/ml tetracycline; E. coli cells containing the plasmid R831b was cultured on solid LB media containing 12.5 μg/ml kanamycin and grown overnight at 37° C.

Chloroplast Vector Constructions

To amplify the merB gene from the native plasmid, a primer pair was designed to have a PstI restriction site followed by a chloroplast and bacterial functional RBS (ribosome binding site) of sequence GGAGG in the 5′ primer, followed by a 4 nucleotide spacer region upstream of the start codon. This primer had 20 nucleotide homology with the 5′ end of the gene and a total of 35 nucleotides. The 3′ primer was designed to have 20 nucleotide homology with the 3′ end of the gene and a ClaI restriction site. To amplify the merA gene from the native plasmid, a 5′ primer was designed to have a ClaI restriction site followed by the RBS sequence and a 4 nucleotide spacer region before the start codon and the 20 nucleotide homology with the merA gene. All primer pairs were designed using the QUICKPRI program of the DNASTAR software. Two PCR reactions were done to amplify the merA and the merB genes individually from the plasmid NR1 that contained the complete and functional merA gene and the plasmid R831b, that contained the full-length merB gene. The PCR products were cloned into suitable plasmid vectors.

pLDR-MerAB-3′UTR Vector Construction

The functional merAB operon was amplified via PCR from the vector pCR2.1-MerAB and a new set of primers was made. The 5′ primer was designed to have an EcoRV site, a RBS (ribosome binding site), a spacer region of 4 nucleotides (attt) and 20 bases of homology to the merAB operon starting at the start codon (atg). The 3′ primer is a simple primer with 20 bases of homology to the 3′ end of the operon. After cloning, correct orientation was verified by restriction analyses.

Mercury Resistance Bioassay in Bacteria

The bacterial clones pLDR-MerAB, pLDR-MerAB-3′UTR and the control E. coli XL1-blue cells were grown for 24 hours at 37° C. in 50 ml LB broth with concentrations of HgCl₂ of 0, 25 μM and 50 μM. The growth medium was autoclaved and cooled to 40° C., before adding HgCl₂ and mixed thoroughly to provide an even concentration throughout the plate or growth medium. The bacterial clones pLDR-MerAB, pLDR-MerAB-3′UTR and the untransformed control E. coli cells were plated in solid LB medium containing HgCl₂ concentrations of: 0, 50 μM, 100 μM, 500 μM. Plates were incubated for 24 hours at 37° C.

Bombardment and Selection of Transgenic Plants

The steps involved in the gene delivery by particle bombardment and the selection process of the transgenic Nicotiana tabacum var Petit Havana clones were performed essentially as describe by Daniell (1997). Tobacco leaves were bombarded using Bio-Rad PDS-1000/He biolostic device. After bombardment, leaves were placed on RMOP medium containing 500 μg/ml spectinomycin for two rounds of selection on plates and subsequently moved to jars on MSO medium containing 500 μg/ml spectinomycin.

Confirmation of Chloroplast Integration by PCR

Plant DNA was isolated using the Qiagen DNeasy Plant Mini Kit (Qiagen, Valencia, Calif.). The PCR primer pairs 3P-3M and 5P-2M were used to confirm the integration of the gene cassette into the chloroplast and the presence of the genes of interest respectively essentially as described elsewhere (Guda et al., 2000). PCR analysis was performed using the Perkin Elmer Gene Amp PCR System 2400 (Perkin Elmer, Chicago, Ill.).

Southern Blot Analysis

The total plant DNA was obtained from transgenic T₀ and T₁ plants as well as from untransformed tobacco plants following the protocol previously explained (Daniell et al., 2001). The plant DNA was digested with BglII and separated on a 0.8% agarose gel at 50 V for 2 hours. The gel was soaked in 0.25 N HCl for 15 minutes and then rinsed 2× with water. The gel was then soaked in transfer buffer (0.4 N NaOH, 1 M NaCl) for 20 minutes and transferred overnight to a nitrocellulose membrane. The membrane was rinsed twice in 2×SSC (0.3 M NaCl, 0.03 M Sodium citrate), dried on filter paper, and then cross-linked in the GS GeneLinker (BIO.RAD). The flanking sequence probe was obtained by BglII/BamHI digestion of the plasmid pUC-ct that contains the chloroplast flanking sequences (trnI and trnA genes). The merAB probe was obtained by EcoRI digestion of plasmid pCR2.1-MerAB. Probes were labeled with ³²P using Ready Mix and purified by using Quant™ G-50 Micro columns (Amersham, Arlington Heights, Ill.), followed by radioisotope incorporation. The probe was quantified by using a Beckmann LS 5000TD scintillation counter. Both prehybridization and hybridization were done using the Quick-Hyb solution (Stratagene, La Jolla, Calif.). The membrane was washed twice in 2×SSC with 0.1% SDS for 15 minutes at room temperature, followed by two additional washes in 0.1×SSC with 0.1% SDS for 15 minutes at 60° C. (to increase the stringency). Blots were exposed to X-ray films and developed in a Konica SRX-101A.

Northern Blot Analysis

The RNeasy Mini Kit and protocol was used to isolate total RNA from plant tissues (QIAGEN, Inc.). The merA, merB, aadA and merAB probes were used to probe different RNA blots. The merA probe was made by cutting out the merA gene from the pCR2.1-MerA vector with EcoRI. The merB probe was made by cutting out the merB gene from the pCR2.1-MerB vector with EcoRI. The aadA probe was amplified by PCR from the pLD-ctv vector with a specific primer pair (5′: ccatggcagaagcggtaatcg/3′:aagatttatttgccgactacctt). The merAB probe was made digesting the pCR2.1-MerAB vector with EcoRI. Restriction fragments were cut out and eluted from the gels. The probe labeling reaction, pre-hybridization/hybridization steps, membrane washing step and autoradiography were performed as explained in the Southern blot section in the materials and methods.

Phenyl Mercuric Acetate Treatments

Seeds of wild type (Nicotiana tabacum var Petit Havana) and two transgenic lines (pLDR-MerAB and pLDR-MerAB-3′UTR) were surface-sterilized in 7% sodium hypochlorite containing 0.1% Tween 20. Seeds were kept on a rocking platform for 20 min, and rinsed in sterile distilled water at least three times. Sterilized seeds were transferred to plates containing half strength MS medium (Murashige and Skoog, 1962) with 0.5 mg/mL spectinomycin, 0.3% phytoagar, pH 5.7. Plates were incubated in the dark at 4° C. for three days, then maintained in a controlled growth chamber at a temperature of 22-24° C., humidity of 75/90% and photon flux density of 750 μE.m⁻² supplied over a 16-h day length. Following germination (after ˜10 days), seedlings were transferred to soil (50:50 sand and Davis Mix) in the greenhouse at 22° C. using a 16-h photoperiod. Five replicate pots each contained a single seedling, either wild type or transgenic plant. All pots were watered twice a week with half strength Hoagland's solution.

Effect of PMA on Seedling Germination

In order to determine the inhibitory concentration of PMA on seedling germination, three different concentrations of PMA were applied to pots containing 16-day old plants from wild type and two transgenic lines in 3 replicates. PMA stock solutions were prepared as 10 mM in dimethyl sulfoxide (DMSO). Different PMA concentrations (50 to 200 μM) were added to each pot in 100 mL of half strength Hoagland's solution. Control pots received the same volume of Hoagland's solution without PMA. All plants were grown in the greenhouse under the same conditions as described above.

Effect of PMA on Potted Plants

Pots of 5 replicates representing the wild type and the two transgenic lines (of approximately the same size) were transferred to PVC plastic trays 3 inches high. Different concentrations of PMA (μM) were prepared (100, 200, 300, and 400) using a stock solution in half strength Hoagland's solution. For each treatment, a single tray maintained approximately 200 mL (to about half of the pot's height) of the PMA-Hoagland's solution. This semi-hydroponic system insures common source of feeding solution and avoids variations in irrigation of individual pots. Moreover, all plants in the same treatment were exposed to exactly the same concentration of PMA. The control tray was filled with half-strength Hoagland's solution without metal. After about 14 days, plants were harvested, washed thoroughly with distilled water, and the length of the longest root and shoot of the plants were measured. Shoots and roots were separated and dry weight was determined.

Determination of Chlorophyll Content in Leaf Disks Treated with PMA

Leaf disks were cut out with a cork-borer (15-mm diameter) from the youngest and fully expanded leaves on 3 week-old plants grew in the soil with no PMA. Disks of wild type and different transgenic plants were placed in Petri dishes containing solidified MS medium (pH 5.7 with no sucrose) supplemented with different concentrations of PMA ranged from: 0.1-1 μM, 10-100 μM, and 200-500 μM. Plates with no PMA were used as controls. The effect of Hg-stress was assessed by the loss of chlorophyll in leaf disks. Leaf disks were collected after 6 days of exposure to PMA. They were immediately extracted in 80%-chilled acetone for determination of total chlorophyll content following the protocol from Current Protocols in Food Analytical Chemistry.

### ILLUSTRATIVE EXAMPLE 2

In Example 1, we successfully integrated, for the first time, both bacterial merA and mer B operon into the chloroplast genome of tobacco plant in a single transformation event. Such engineered plants provide important means of removing Hg from contaminated environments by metabolizing the toxic form of Hg (e.g. PMA) into less toxic and volatile Hg form (e.g. Hg [0]). The bacterial merB gene, encodes an organomercurial lyase, degrades MeHg to methane and Hg [II] while the bacterial merA gene, coding for the mercuric ion reductase (merA), converts ionic mercury (Hg [II]) to the volatilized elemental mercury (Hg [0]). The results of the transgenic lines showed that the plants expressing both genes were capable of tolerating up to 400 μM of organomercurials compounds when grown in soil.

Our previous results on the transgenic tobacco overexpressing the mer operon raised a number of questions of particular importance to their use in phytoremediation. For instance, from a phytoremediation perspective, it is necessary to determine if the transgenic tobacco accumulates more Hg than the wild type and if they translocate this Hg more efficiently from roots to shoots. Additionally, we hypothesized that inserting the mer operon into the chloroplasts may confer to the transgenic plants increased ability to convert Hg to less toxic, volatile forms.

In the present study we extended our previous work by characterizing the physiological differences between wild type (WT) and two transgenic tobacco lines overexpressing the merA and merB genes in the chloroplast of tobacco plant. The specific objectives are: i) to examine the uptake of Hg by transgenic tobacco plants grown in soils amended with mercuric chloride (HgCl₂) as an inorganic form and phenylmercuric acetate (PMA) as an organic form, ii) to determine the efficiency of the transgenic lines to translocate Hg from roots to shoots and the forms in which Hg accumulated in plant tissue, iii) to examine the ability of mer A and B transgenic lines to volatilize Hg [0].

Material and Methods

Construction of Tobacco Plant Containing Organomercurials and Mercury Resistance Genes

As described in Example 1, the bacterial native genes, merA (1.69 kb) and merB (638 bp) were integrated into a chloroplast vector. This vector allows site-specific integration of the transgene in the inverted repeat regions of the chloroplast genome between the trnI and trnA genes by homologous recombination. The constitutive promoter (Prrn) drives the transcription of the downstream genes that include the aadA (aminoglycoside 3′-adenylyltransferase) gene which confers resistance to spectinomycin and the mer A and B operon. Two versions of the chloroplast vector were made with the presence or absence of the 3′untranslated region (UTR) from the chloroplast psbA gene that was anticipated to confer stability to the transcripts. The chloroplast transgenic tobacco were obtained with each construct and they were designated as pLDR-MerAB-3′UTR and pLDR-MerAB respectively.

Plant Germination in Contaminated Soil

Tobacco seeds of both transgenic lines and WT were surface-sterilized by shaking in 70% ethanol for 30 seconds, followed by 10% sodium hypochlorite for 30 minutes, and five 5-minute washes with sterile double-distilled water. During the sterilization procedure, the seed were kept in closed, sterile plastic tubes and shaken on a rocking platform to ensure the bleach and alcohol covered all of the seed. As described in Ruiz et al. (2003), sterilized seeds were transferred to plates containing half strength MS medium (Murashige and Skoog, 1962) with 0.5 mg/mL spectinomycin, 0.3% phytoagar, pH 5.7 and no sucrose was added. Plates were incubated in the dark at 4° C. for three days, and then were maintained in a controlled growth chamber with temperature (22-24° C.), humidity 75/90% and light (750 μE.m⁻²) with 16 h day length. Ten-day old seedlings were transferred to soil (50:50 sand and Davis Mix) in the greenhouse at 22° C. using 16 h of light. Each pot contained a single seedling, either WT or transgenic plant. All pots, 5 replicates of each line, were watered twice a week with half strength Hoagland's solution for 10 days.

Different concentrations of phenyl mercuric acetate (PMA) and mercuric chloride (HgCl₂) were added to each pot at 100, 200, and 300 μM using a stock solution in half strength Hoagland's solution. The control pots were watered with half-strength Hoagland's solution without metal. After about 15 days, the plants were harvested, washed thoroughly with distilled water, and the length of the longest root and shoot of the plants were measured. Shoots and roots were separated and immersed directly in liquid nitrogen. The frozen plants were dried using freeze dryer and the dry weight was determined. Samples were stored at −80° C. for chemical analysis.

Determination of Total Mercury Concentration in Plant

Freeze dried samples were ground to a fine powder using liquid nitrogen and analyzed via automated CVAAS (Cold Vapor Atomic Absorption Spectroscopy) with a continuous flow Vapor Generation Accessory, VGA-77 as described in Varian Operation Manual (Hams, 1997).

Plant samples were acid-digested by stepwise additions of 70% (w/v) nitric acid, 30% (w/v) hydrogen peroxide, and concentrated HCl at 95° C. in a modification of EPA method 3010A (1992). Potassium permanganate (2%) and potassium persulfate (5%) were added to the samples before addition of nitric acid to reduce organic mercury in the digestion solution (Munns and Holland, 1971). After digestion, the excess potassium permanganate was reduced with hydroxylamine hydrochloride. Blanks and standard reference materials of San Joaquin soil (SRM 2709), National Institute of Science and Technology (1.4 μg of Hg g⁻¹) were run as external quality controls for analyses of Hg in soil and plant samples.

Measurement of Hg Volatilization

Volatilize Hg[0] was measured from the other three replicates of each treatment (PMA and HgCl₂). Pots (each contained a single plant) were placed in 3-L volume gastight acrylic volatilization chambers (FIG. 1), in the greenhouse, through which a continuous air flow (1.5 L min⁻¹) was passed by applying suction at the outlet and by bubbling incoming air into the oxidizing trap solution. The background Hg volatilization from the soil was obtained from chambers include pots without plants using the same treatments as above. The actual plant volatilization was determined by subtracting the total volatilization from the background values.

Volatile Hg was quantitatively trapped in alkaline peroxide liquid traps composed of 0.1% NaOH and 30% H₂O₂ (1:1) as described previously (Zayed and Terry, 1992, Zayed et al., 1998). Aliquots (10 mL) of trap solution were collected every 24 h, after which the solutions were replaced successive for 13 days. The trap solution samples were heated in at 95° C. to remove the peroxide. The Hg concentration was measured by vapor-generation atomic absorption spectroscopy as described above.

Speciation of Hg by X-ray Absorption Spectroscopy (XAS)

Because of the non-destructive nature of X-ray absorption spectroscopy (XAS), it provides element-specific structural information detailing the atomic arrangement of unknown elements in situ (Lee et al., 2001, Riddle et al., 2002). This allows for independent analysis of Hg species in complex mixtures that may be found in plant tissues. Conventional speciation techniques involving sequential extraction may, by the very nature of the process, disturb or chemically alter the chemical form of Hg in the sample. XAS analysis, on the other hand, directly identifies the chemical species present, without chemical modification by extractants.

Individual plants (WT and transgenic plants) from each treatment were pooled into one shoot or root sample, immediately frozen in liquid nitrogen, ground, and stored at −80° C. for XAS analysis. Frozen plant specimens were carefully packed into 2-mm path-length lucite sample holders with mylar tape for windows, and kept under liquid nitrogen.

XAS analysis was conducted at beam line 4-3 at the Stanford Synchrotron Radiation Laboratory (SSRL). Si(220) double crystal monochromator was used with an upstream vertical aperture of 1 mm, and harmonic rejection was achieved by detuning one crystal by 50%. The source electron energy was 3.0 GeV with a current ranging from 60 to 100 mA. Samples were positioned at a 45° angle to the X-ray beam and were maintained at 15 K in a flowing liquid helium cryostat. X-ray absorption spectra were collected by monitoring the mercury L3 edge (12284.4 eV by calibration with the Au L3 edge) fluorescence using a Canberra 13-element Ge detector, in a series of replicate scans. Spectra were also collected for standard reference materials of Hg and energy was calibrated by using the spectrum of Hg[0]. Data were collected and analyzed using the program suites XAS-Collect (George et al. 2000) and EXAFSPAK, respectively.

Quantitative analysis was carried out using an edge-fitting method (Pickering et al., 1995), in which the normalized edge spectrum of a sample containing unknown Hg species is fit to a linear combination of the spectra of standard Hg compounds by using a least-squares minimization procedure. The fractional contribution of a standard spectrum to the fit is equivalent to the fractional abundance of Hg in that chemical species in a sample. In the present work, simultaneous measurement of the X-ray absorption edge spectrum of standard solutions of HgCl₂ and PMA were chosen to be representative of potential Hg species present. The precision of the determined fractional abundances of Hg chemical species is 3 times the estimated standard deviations (calculated from the diagonal elements of the variance-covariance matrix) and is equivalent to the 95% confidence limits. The accuracy of the values obtained depends upon the degree of similarity between the standard spectra chosen and their counterparts in the plant spectra, and is generally larger than the precision.

Statistical analyses were performed with Costat statistical package (SAS Institute, Cary, N.C.) using unpaired t-test and analysis of variance procedures.

Results showing Plant Growth and Tolerance

The mercury tolerance of wild type (WT) and transgenic plants was tested by treatment with phenyl mercuric acetate (PMA), an organic form of mercury, or mercuric chloride (HgCl₂), an inorganic form of mercury.

The dry weight of shoots and roots of WT and transgenic lines were measured for each PMA and HgCl₂ treatment (FIG. 2, 3). With PMA, the dry weights of WT shoots and roots decreased progressively as PMA concentration was increased from 0 to 300 μM (FIG. 2). Although the dry weights of the roots and shoots of the transgenic lines also decreased with increasing PMA concentration, the reduction in growth was less than that of WT and the transgenic lines grew substantially better than WT at concentrations of PMA in the soil (FIG. 2). These results are consistent with our previous results (Ruiz et. al., 2003). On the other hand, treatment with HgCl₂ reduced plant dry weights less than treatment with the same concentration of PMA (FIG. 3). Increasing the concentration of HgCl₂ from 0 to 300 μM, led to no significant differences between the dry weights of WT and transgenic lines (FIG. 2), although the dry weights of the transgenic lines were, on average, than that of WT plants (FIG. 2). Since the transgenic lines have 2-fold dry weights than WT under PMA treatment, our results indicate that the overexpression of the mer operon in plant plastids increased the resistance of the plants to highly toxic organomercurials.

To compare the resistance of the WT and transgenic plants to the toxic effects of mercury, we used root length as a reliable parameter for heavy metal tolerance (Murphy and Taiz 1995). After a 15-day growth period on soil containing 100, 200, or 300 μM PMA or HgCl₂, roots of the transgenic plants were significantly longer (P<0.05) than those of WT plants (FIG. 2, 3). For example, root lengths of both transgenic lines were reduced an average of 4 cm when the PMA concentration was increased from 0 μM to 300 μM, while WT root lengths were reduced an average of 6 cm at the same concentrations (FIG. 2). On the other hand, increasing HgCl₂ concentration in the soil from 0 to 300 μM increased the roots lengths of the transgenic lines compared to WT, which was not significantly affected by increasing the HgCl₂ concentration. As demonstrated in the previous work, the presence of the 3′untranslated region (UTR) in the chloroplast vector from the chloroplast psbA gene had no significant effect on plant growth under any of the mercury treatments (FIG. 2, 3).

Hg Accumulation:

The total mercury accumulation in plant roots and shoots was determined for the WT and transgenic plants treated with PMA or HgCl₂. The results show that Hg concentrations reached thousands of ppm in roots compared to tens of ppm in shoots of the plants when supplied with either PMA or HgCl₂ (FIG. 4).

Transgenic plants accumulated significantly amounts of mercury in their roots than did WT when treated with the same concentrations of PMA (100 and 200 μM PMA (P<0.05); 300 μM PMA (p<0.001); (FIG. 4-A)). When grown on soil amended with HgCl₂, the increase in the total mercury accumulation in roots of transgenic lines versus WT was highly significant (P<0.001) at 300 μM but not significant at 100 and 200 μM (FIG. 4-B). Transgenic plants grown on soil with lower mercury concentrations (100 and 200 μM), mercury concentrations in their roots were ranging from 450 to 600 ppm. At higher treatment concentrations (300 μM), mercury concentrations in the roots reached up to 1500 ppm (FIG. 4-A, B). Roots of WT, on the other hand, mercury concentrations were not significantly affected by increasing the external concentration of PMA or HgCl₂ from 100 to 300 μM in the soil (FIG. 4-A, B).

In transgenic shoots, Hg concentrations were highly significantly greater (P<0.001) than WT in the transgenic plants at all PMA concentrations tested (100 to 300 μM) (FIG. 4-C). With HgCl₂ treatment, the mercury concentration in shoots of transgenic plants was significantly higher at 100 and 200 μM than in WT shoots treated with the same concentrations (FIG. 4-D). However, treatment with 300 μM HgCl₂ significantly reduced mercury concentrations in transgenic shoots compared to the same lines grown at 100 and 200 μM but had no significant effect on Hg accumulation in WT shoots (FIG. 5D).

Interestingly, the accumulation of mercury in plant tissues was also affected by increasing the mercury concentration in the soil. For example, at 100 and 200 μM PMA the shoots of transgenic plants accumulated the average of 32 times more mercury than WT, while at 300 μM this accumulation increased up to 95 times compared to WT (P<0.001) (Table 1). However, when HgCl₂ was supplied, shoots of transgenic plants accumulated 3 times more at 100 μM and 9 times more at 200 μM mercury compared to WT shoots, but mercury accumulation drastically decreased significantly in shoots of both WT and transgenic lines when treated with 300 μM HgCl₂ such that there were no significant differences between the lines (Table 1). This result demonstrates that the overexpression of mer operon in the chloroplasts of tobacco plants enhances the uptake of organic mercury by the plant roots and significantly increases the translocation of Hg from root to shoot.

Hg Speciation (XAS-analysis)

To investigate the chemical forms of mercury accumulated in shoots and roots of plants exposed to PMA or HgCl₂, XAS was performed on shoots and roots of transgenic plants treated with 100 μM PMA or HgCl₂ for 15 d. The representative Hg L3 near-edge X-ray absorption spectra of PMA and HgCl₂ references are shown in FIG. 5-A. The spectra of the plant samples were fitted to the reference spectra (FIG. 5 B-C). It is clear that the predominant form of mercury accumulated in the tissues of the transgenic plants depends on the form of mercury supplied. For example, the spectra of shoots and roots from PMA-supplied plants were very similar to each other and to the organic reference (FIG. 5-B). When plants were supplied with HgCl₂, the spectra of shoots and roots were similar to that of the inorganic reference (FIG. 5-C). Curve fitting analysis indicated that, when PMA was supplied, mercury accumulated by the plant was approximately 82% organic Hg-like and 18% of inorganic Hg-like form. In contrast, HgCl₂-supplied plants accumulated 77% of total mercury in an inorganic Hg form and 33% in an organic-like form (FIG. 6). As mentioned above, the mercury content in the WT plants was significantly lower than in the transgenic lines when supplied with either PMA or HgCl₂ (FIG. 4). The X-ray absorption spectroscopy edge-fitting results for WT indicate that the mercury accumulated by WT plants is entirely in the form of the mercury supplied.

Hg Volatilization

To determine the effect of the overexpression of the mer operon gene on the production of volatile mercury, Hg [0] volatilization (μg Hg volatilized g⁻¹ dry weight d⁻¹) was measured from WT, and the pLDR-merAB and pLDR-merAB 3′UTR transgenic lines continuously over a 13-day period after treating the soil with 100 μM PMA or HgCl₂. The background mercury volatilization was measured in treated soil with no plants and was subtracted from the values obtained for plants treated with mercury.

The results indicated that the volatilization rate of elemental mercury (added as PMA or HgCl₂) was substantially higher for genetically engineered plants than the WT (FIG. 7). When plants were supplied with PMA, rates of mercury volatilization reached a maximum at 2 days after the treatment began. By 5 days, rates of volatilization had diminished to background levels. In the case of plants supplied with HgCl₂, volatilization reached a maximum 3 days after treatment began and reached background levels at 6 or 7 days after treatment began. The WT plants failed to volatilize mercury under each treatment; the amount of elementary mercury evolved from WT chambers was nearly the same as that of background Hg from the chambers with no plants (FIG. 7).

During the active volatilization period, the rates of Hg[0] volatilization were 6 times higher for the pLDR-merAB plants supplied with PMA compared with HgCl₂ (FIG. 7-A). On the contrary, merAB-3′UTR plants volatilized 2 times more Hg[0] when supplied with 100 μM HgCl₂ than with same concentration of PMA (FIG. 7-B).

Discussion

The two bacterial mer operon genes, merA and merB, were expressed in the chloroplasts of tobacco plants to degrade the toxic forms of mercury in two-step conversion to less toxic-volatile Hg[0] as described in Example 1. The results clearly show that the overexpression of merA and mer B genes in the plastids of tobacco plants is suitable for the phytoremediation of mercurial and organomercurials compounds from the contaminated soils. The transgenic plants were able to tolerate, take up, and assimilate highly toxic organic mercury to less toxic elemental form better than the wild type (WT). Since HgCl₂ was less toxic to the merAB-tobacco plants than the PMA supplied at the same concentration, the transgenic lines should be able to tolerate even higher concentrations of HgCl₂ encountered in contaminated sites.

By using root lengths to measure Hg tolerance, we showed that the transgenic lines are less affected by the presence of toxic concentrations of Hg than the WT. Diverse plant species overexpressing foreign genes have shown high resistance to heavy metals by increased root length. For example, Zhu et al., (1999) reported that overexpression of γ-glutamylcysteine synthetase (γ-ECS) in the plastids of Indian mustard enhances Cd tolerance as demonstrated by the fact that their root lengths were 2-fold longer than the WT. In addition to the significant results observed for root lengths, in the present study, the total dry weights of the transgenic plants were significantly higher in PMA-supplied plants compared to HgCl₂. These results indicated undoubtedly that the transformation of bacterial merA and merB genes into tobacco chloroplasts increased the resistance of the transgenic plants to the highly toxic effects of PMA. These results are consistent with those of Heaton et al., (1998) who showed that nuclear engineered tobacco plants expressing the reconstructed bacterial mercuric ion reductase gene (merA9) were able to grow much better on soils containing 100 and 500 ppm Hg(II) than WT, which quickly died.

As expected, accumulation of mercury in shoots is much lower than in the roots. Shoots of transgenic plants were found to accumulate 5-16 ppm compared to 500-1500 ppm in roots. This ratio of accumulation is consistent with other results of Riddle et al., (2002) in which shoots of water hyacinths plants accumulated about 70 ppm Cr and 0.20 ppm Hg compared to 5500 ppm Cr and 16 ppm Hg in roots, respectively. They suggested that mercury is bound in the roots by ionic carboxylate binding whereas in the shoots it is bound in covalent Hg-S binding. (because GSH is a major component of the active oxygen scavenging system of the cell (Li et al., 1997, Noctor and Foyer, 1998), it is possible that the levels of GSH may have contributed to increased heavy metals tolerance by protecting cells from metal-related oxidative stress damage (Gallego et al., 1996; Weckx and Clijsters 1996, 1997).

Since the shoot is the most easily harvestable part of the plant, an effective phytoextraction technology requires the accumulation of high concentrations of trace elements in above-ground biomass (Krämer and Chardonnens, 2001). Interestingly, our results indicated that inserting the mer operon into the chloroplasts to enhance the phytoextraction of mercurial compounds maximized root-to-shoot transport. The shoots of transgenic lines were able to accumulate 35 times more Hg than WT when treated with 100 or 200 μM PMA. At 300 μM, the transgenic lines accumulated 90 times more mercury. With 300 μM HgCl₂ treatment, the transgenic lines accumulated 4 times more than the WT. These results were confirmed with high rates of volatilization from the transgenic plants compared to no volatilization from the WT. The hyperaccumulator plants exhibit an exceptionally high ratio of shoot vs root metal concentrations, as well as extreme metal tolerance (Kramer and Chardonnens, 2001). X-ray absorption spectroscopy analysis showed that organic PMA-like species represented the major component (about 83%) of total Hg accumulated in the plants shoots and roots of transgenic plants supplied with organic mercury (PMA). In contrast, when plants were supplied with inorganic mercury (HgCl₂), plants accumulated mercury predominantly in an HgCl₂-like form. Although the relative amounts of organic and inorganic Hg species accumulated depended upon the Hg species supplied, the presence of both indicates that the expression of mer A and mer B in tobacco plants allows for the conversion of both Hg species to Hg[0] and may account for the elevated rates of mercury volatilization observed from the transgenic plants. On the other hand, XAS revealed that none of the Hg accumulated in the tissues of WT plants treated either by PMA or HgCl₂ converted into the other form. Thus, plants expressed merA and mer B genes in the plastids have enhanced potential for the detoxification of different forms of mercury compared to WT plants.

Such higher translocation of mercury from roots to shoots of the transgenic plants was also reflected on the enormous mercury volatilization. The kinetics of Hg volatilization by merAB plants was monitored continuously after the soil was treated with 100 μM PMA or HgCl₂. The higher rate of elemental mercury evolution from the transgenic plants conformed that the expression of mer B and mer A genes increase the conversion of methylmercury, the form taken up by plant roots, to sulfhydryl-bound Hg(II) and the conversion of ionic mercury to volatile Hg[0], which is released into the atmosphere.

Since tobacco is a fast growing plant species capable of growth in different environmental conditions with an extensive root system and large and broad leaves, it is considered as an excellent crop for phytoremediation. In this work, transforming the tobacco plastids with foreign bacterial mer A and mer B genes has greatly increased the potential of this multiple use crop to remediate mercury-contaminated environments.

### ILLUSTRATIVE EXAMPLE 3

The overall objective of this project is to engineer transgenic microalgae, a unicellular, photosynthetic aquatic plant, capable of the removal and degradation of explosive and nitroaromatic contaminants from contaminated water in bioreactor systems. The microalgae will also be engineered to sequester and detoxify heavy metals, common toxic copollutants of explosives. Fundamental and applied studies will be undertaken to characterize and develop microalgae as tools for phytoremediation. By the end of the project we aim to have pilot scale bioreactors available which contain microalgae expressing transgenes for the detoxification of nitrate ester, nitroaromatic and nitramine classes of explosives and the sequestration of heavy metals.

Background and Methodology

Example 3 incorporates all of the procedures, protocols and methods described in Examples 1 and 2.

Environmental contamination by explosives is a serious problem, as a vast amount of land and water worldwide is contaminated with persistent explosive residues due to the manufacture, disposal, and testing of munitions. Explosives and their derivatives are highly toxic and as such require remediation to prevent adverse effects on human and environmental health. Many of these compounds are found as co-contaminants often in association with other toxic pollutants such as heavy metals. This presents an even greater remediation challenge. To date, remediation has been attempted mainly through the economically and environmentally costly large-scale removal of soil and water. An alternative is urgently required and much work has focused on microbial biodegradation, but the effectiveness of such approaches has been inhibited by factors such as poor biomass, the requirement of additional substrates, the generation of toxic metabolites and the presence of toxic heavy metal co-contaminants. Such co-contaminants have an inhibitory effect on the biological activity catalysing the remediation processes.

Over recent years bacteria have been isolated which can utilize all the major classes of explosives as nitrogen sources for growth. The genes encoding the enzymes responsible for the bacterial degradation of nitrate esters, nitroaromatic and nitramine explosives have been cloned. Work undertaken by Dr. Rosser has demonstrated that these genes can be expressed in plants, vastly enhancing the plants' ability to tolerate and degrade explosives (Rosser et al, 2001, In Vitro Cellular and Developmental Biology-Plant 37: 330-333; Hannink et al, 2001 Nature Biotechnology, 19: 1168-1172; French et al, 1999, Nature Biotechnology, 17: 491-494). This work has led to fascinating insights into how innate plant enzymes conjugate the metabolites of TNT and store them in a non-extractable, non-toxic form. This is in contrast to bacterial transformation, which results in the toxic TNT intermediates being released back into the media and which is not ideal for a bioremediation system. More recently a strain of Rhodococcus sp. was isolated which can completely degrade RDX. The gene that encodes this activity has been cloned and sequenced and found to be a cytochrome P450 (Seth-Smith et al, 2002, Applied and Environmental Microbiology, 68: 4764-4771). Additionally, genes coding for phytochelatin synthase have been recently isolated from wheat and Arabidopsis. This enzyme performs an important function in plants by producing small peptides, phytochelatins, in response to heavy metal exposure, allowing plant cells to sequester heavy metal ions. We are therefore in the exciting position of having a range of genes available which, when expressed in plant systems, can detoxify all of the major classes of explosives and associated heavy metal contaminants.

While these genes are being exploited for the potential remediation of contaminated soil using terrestrial plants the problem of contaminated groundwater and wastewater originating from the manufacturing and decommissioning of explosives still remains. This example illustrate the potential use of microalgae bioreactor systems to remediate explosive-contaminated waters. This example combines the advantages of bacterial explosive degrading transgene expression in a plant cellular environment for use in an aquatic system.

Photosynthetic algae are the dominant producers in aquatic environments, accounting for substantial oxygen production and carbon dioxide fixation. Chlorella, a unicellular green alga, can serve to remediate polluted aquatic environments as well. Water from these environments can pass through a packed-bed bioreactor in which Chlorella cells grow and be detoxified as the algal cells phytoremediate. The ability of these cells to phytoremediate can be enhanced by the addition of exogenous genes to the cells' chloroplast genome. When expressed, these transgenes allow Chlorella cells to have increased phytoremediant abilities. Through microprojectile bombardment, Chlorella incorporates exogenous genetic material and stably expresses it (Dawson et al, 1997, Current Microbiology 35: 356-362). This material can be inserted into a chloroplast transformation vector designed specifically for homologous recombination with the chloroplast genome. The vector itself contains sequences found in a transfer RNA gene cluster of the chloroplast genome; these sequences surround a gene for streptomycin resistance as well as one for green fluorescent protein (GFP) expression. Thus, resistance to this antibiotic as well as GFP fluorescence indicates stable incorporation of transgenes into the chloroplast genome. Additionally, approximately 80 copies of the chloroplast genome exist in each cell, meaning that overexpression of transgenes from multiple genomic copies is possible. Multiple copies of transgenes may be integrated at several intergenic spacer regions within the chloroplast genome. Chloroplast transformation has been repeatedly shown to produce 500-1000 fold more proteins or enzymes than nuclear genetic engineering (Daniell, et al, 2002, Trends in Plant Science 7: 84-91).

Preliminary studies on wild-type Chlorella. The ability of Chlorella to tolerate uptake and transform TNT, its nitroaromatic derivatives and RDX will be investigated using sterile media containing varying concentrations of energetic or nitroaromatic material. The effect of RDX, TNT and breakdown products on the physiology of Chlorella will be evaluated. The breakdown products resulting from the activity of innate Chlorella detoxification enzymes will be characterised and compared to those obtained in higher plants and bacterial systems. Attempts will be made to identify the innate enzymes involved in the transformation and detoxification processes. Preliminary studies indicate that Chlorella can tolerate cadmium sulfate levels of at least 200 μM. Phytochelatins synthesized as a result of heavy metal exposure can be identified through HPLC analysis. The information gained in these studies will provide a valuable insight into what happens in the natural environment and may provide target enzymes for activity enhancement in future phytoremediation processes.

Generation and characterization of transgenic Chlorella expressing bacterial nitroreductases. Expression of the bacterial gene onr encoding PETN reductase in tobacco resulted in plants that demonstrated a profound increase in their ability to detoxify and degrade the explosive nitroglycerin. PETN reductase also displays activity towards TNT and the transgenic plants expressing this enzyme showed the ability to tolerate and detoxify low levels of TNT that is particularly phytotoxic. Previous work has also resulted in the construction of tobacco plants expressing the aromatic nitroreductase from the bacterium Enterobacter cloacae. These plants showed a remarkable increase in the ability to tolerate TNT and remove it and its metabolites from media at saturating concentrations. In order to enhance the ability of Chlorella to transform nitroaromatic contaminants the bacterial genes PETN reductase and aromatic nitroreductase will be expressed in Chlorella Preliminary studies suggest that when bacterial nitroreductase enzymes are expressed in plant cells, TNT and DNT are transformed and sequestered conjugated with sugars, glutathione, amino acids or malonic acid as part of the detoxification process. Mass balance, toxicity and transformation studies will be performed to determine the levels of TNT and other nitroaromatic compounds that the transgenic Chlorella can tolerate and remove from the media. Work will be carried out to determine the fate of the transformation products and how closely they are related to the products and processes occurring in plants.

Engineering Chlorella to degrade RDX. The bacterial P450 enzyme from Rhodococcus sp 11Y (exp1A) has recently been shown to completely degrade RDX to nitrite, ammonia, formaldehyde, formate and nitrous oxide. This novel P450 will be expressed in Chlorella in order to enhance its capacity to breakdown RDX. Preliminary results also show that a second P450, the mammalian P450 3A4, also has the ability to degrade RDX and uniquely the nitramine explosive HMX. These two P450 genes will be expressed in Chlorella and the transgenic algae investigated for enhanced capability to tolerate and degrade nitramine explosives. Studies have shown that plants tend to accumulate but not transform RDX. It is hoped that by expression of these transgenes that Chlorella will acquire the ability to mineralise RDX and HMX. The transgenic Chlorella will be grown in media containing varying concentrations of energetic compounds. Uptake mass balance and metabolism studies of RDX and HMX will be performed on both transgenic lines as described in Examples 1 and 2.

Chlorella expressing both nitroreductase and P450 genes. Since many sites are contaminated with both nitroaromatic and nitramine compounds it will be important to engineer a single system which can degrade/sequester both types of contaminant. RDX is not particularly phytotoxic but in order to overcome the toxicity presented by nitroaromatic contamination it will be essential to express the bacterial nitroreductase gene and a RDX degrading P450 gene simultaneously in a single Chlorella line.

All reference cited to herein and contained in the reference section of this application are fully incorporated by reference into the text of this specification.

### REFERENCES

1. Banuelos, G. S., Terry, N, Zayed, A. and Wu, L. (1995) Managing high soil Se with phytoremediation. In Selenium: Mining, Reclamation, and Environmental Impact (Schuman, G. E., Vance, G. F., eds). Proceedings of the 12th Annual National Meeting of the American Society for Surface Mining and Reclamation. Gillete, Wyo. American Society for Surface Mining and Reclamation, Princeton, W. Va., pp 394-405.
2. Begley T P, Walts A E, Walsh C T (1986) Bacterial organomercurial lyase: overproduction, isolation, and characterization. Biochemistry 25: 7186-7192.
3. Bendich, A J (1987) Why do chloroplasts and mitochondria contain so many copies of their genome? Bioassays 6: 279-282.
4. Bernier M, Popovic R, Carpentier R (1993) Mercury inhibition of photosystem II. FEBS Lett 32: 19-23.
5. Bizily S, Rugh C C, Meagher R B (2000) Phytoremediation of hazardous organomercurials by genetically engineered plants. Nat Biotechnol 18: 213-217.
6. Bizily S, Rugh C C, Summers A O, Meagher R B (1999) Phytoremediation of methylmercury pollution: merB expression in Arabidopsis thaliana plants confer resistance to organomercurial. PNAS 96: 6808-6813.
7. Clemens S. Antosiewicz D M, Ward J M, Schachtman D P, Schroeder J1 (1998) The plant Cdna LCT1 mediates the uptake of calcium and cadmiumin yeast. Proc. Natl. Acad. Sci. USA 95:12043-12048.
8. Cohen C K, Fox T C, Garvin D F, Kochian L V (1998) The role of iron-deficiency stress responses in stimulating heavy-metal transport in plants. Plant Physiol 116:1063-1072.
9. Creissen, G., Firmin, J., Fryer, M., Kular, B., Leyland, N., Reynolds, H., Pastori, G., Wellburn, F., Baker, N., Wellburn, A. and Mullineaux, P. (1999) Elevated glutathione biosynthetic capacity in the chloroplasts of transgenic tobacco plants paradoxically causes increased oxidative stress. Plant Cell, 11, 1277-1291.
10. Cunningham S D, Berti W R (2000) Phytoextraction and phytostabilization: technical, economic, and regulatory considerations of the soil-lead issue. In: Terry N, Bañuelos G (eds) Phytoremediation of contaminated soil and water. CRC Press, Boca Raton, Fla., pp 359-376.
11. Daniell H (1997) Transformation and foreign gene expression in plants mediated by microprojectile bombardment. Methods Mol Biol 62: 453-488.
12. Daniell H (2002) Molecular strategies for gene containment in transgenic crops. Nat Biotechnol 20(6): 581-586.
13. Daniell H (2003) Medical Molecular Pharming: Expression of antibodies biopharmaceuticals and edible vaccines via the chloroplast genome. Plant Biotechnology, 2000 and Beyond, in press.
14. Daniell H and Parkinson L (2003) Jumping genes and containment. Nature biotechnology 21: 374-375.
15. Daniell H, Datta R, Varma S, Gray S, Lee S B (1998) Containment of herbicide resistance through genetic engineering of the chloroplast genome. Nat Biotechnol 16: 345-348.
16. Daniell H, Dhingra A (2002) Multigene engineering: dawn of an exciting new era in biotechnology. Curr Opin Biotechnol 13 (2): 136-171.
17. Daniell H, Khan M. S, Allison L (2002) Milestones in chloroplast genetic engineering: an environmental friendly era in biotechnology. Trends Plant Sci 7 (2): 84-91.
18. Daniell H, Lee S B, Panchal T, Weibe P O (2001) Expression of the native cholera toxin B subunit gene and assembly as functional oligomers in transgenic chloroplasts. J Mol Biol 311: 1001-1009.
19. Daniell H, Muthukumar B, Lee S B (2001) Marker free transgenic plants: engineering the chloroplast genome without the use of antibiotic selection. Current Genetics 39: 109-116.
20. De Gray G, Rajasekaran K, Smith F, Sanford J, Daniell H (2001) Expression of an antimicrobial peptide via chloroplast genome control phytophatogenic bacteria and fungi. Plant Physiol 127: 852-862.
21. de Souza, M. P., Chu, D., Zhao, M., Zayed, A. M., Ruzin, S. E., Schichnes, D. and Terry, N. (1999) Rhizosphere Bacteria Enhance Selenium Accumulation and Volatilization by Indian Mustard. Plant Physiol., 119(2), 565-574.
22. de Souza, M. P., Pilon-Smits, E. A. H., Lytle, C. M., Hwang, S., Tai, J., Honma, T. S. U., Yeh, L. and Terry, N. (1998) Rate-limiting steps in selenium assimilation and volatilization by Indian mustard. Plant Physiol., 117, 1487-1494.
23. Dean J G, Bosqui F L Lanouette V H (1972) Removing heavy metals from waste water. Environ Sci Technol 6: 518-522.
24. DeCosa B, Moar W, Lee S B, Miller M, Daniell H (2001) Hyper-expression of the Bt Cry2Aa2 operon in chloroplasts leads to formation of insecticidal crystals. Nat Biotechnol 19: 71-74.
25. Delhaize, E. (1996). A metal-accumulator mutant of Arabidopsis thaliana. Plant Physiol., 111, 849-855.
26. Doucleff M, Terry N (2002) Pumping out the arsenic. Nat Biotechnol 20: 1094-1095.
27. EPA. Method 3010A (1992) In Methods for Chemical Analysis of Water and Wastes; U.S. Environmental Protection Agency: Washington, D.C.
28. Fernandez-San millan A, Mingo-Castel A, Miller M, Daniell H (2003) A chloroplast transgenic approach to hyper-express and purify human serum albumin, a protein highly susceptible to proteolytic degradation. Plant Biotechnol J, in press.
29. Gallego, S. M., Benavides, M. P. and Tomaro, M. L. (1996) Effect of heavy metal ion excess on sunflower leaves: evidence for involvement of oxidative stress. Plant Sci., 121, 151-159.
30. Gavis, J., Ferguson, J. F. (1972) The cycling of mercury through the environment. Water Res., 6, 989-1008.
31. George, M. J. (2000) XAS-Collect: A computer program for X-ray absorption spectroscopic data acquisition. J. Synchrotron Radiat., 7, 283-286.
32. Glass D J (1999) Markets for phytoremediation, 1999-2000. Glass Associates, Needham, Mass., htt://www.channel1.com/dglassaccoc/index.htm.
33. Guda C, Lee S B, Daniell H. (2000) Stable expression of biodegradable protein base polymer in tobacco chloroplasts. Plant Cell Rep 19: 257-262.
34. Hams, G. A. (1997) Determination of mercury in blood and urine by Cold Vapor AAS using the VGA-&&. AA Instruments at work, Varian operation manual, AA-126, 1-4.
35. Heaton A C P, Rugh C L, Wang N J, Meagher R B (1998) Phytoremediation of mercury and methylmercury polluted soils using genetically engineered plants. J Soil Contam 7: 497-509.
36. Heifetz, P. B. (2000) Genetic engineering of the chloroplast. Biochemie, 82, 655-666.
37. http://www-ssr1.slac.stanford.edu/exafspak.html, Stanford Synchrotron Radiation Laboratory. (2002). EXAFSPAK. SSRL, Palo Alto, Calif.
38. Jackson W J, Summers A O (1982) Biochemical characterization of HgC12-inducible polypeptides encoded by the mer operon of plasmid R100. J Bacteriol 151: 962-970.
39. Ji, G., Silver, S. 1995. Bacterial resistance mechanisms for heavy metals of environmental concern. J Ind Microbiol 14, 61-75.
40. Kärenlampi S, Schat H, Vangronsveld J, Verkleij J A C, Lelie D, Mergeay M, Tervahauta A I (2000) Genetic engineering in the improvement of plants for phytoremediation of metal polluted soils. Environ Pollut 107: 225-231.
41. Ke, H. Y., Sun, J. G., Feng, X. Z., Czako, M. and Marton, L. (2001) Differential mercury volatilization by tobacco organs expressing a modified bacterial merA gene. Cell Research, 11(3), 231-236.
42. Kota M, Daniell H, Varma S, Garczynski F, Gould F, Moar W J (1999) Overexpression of the Bacillus thuringiensis Cry2A protein in chloroplast confers resistance to plants against susceptible and Bt-resistant insects. Proc Natl Acad Sci USA 96: 1840-1845.
43. Krämer U, Chardonnens A N (2001) The use of transgenic plants in the bioremediation of soil contaminanted with trace elements. Appl Microbiol Biotechnol 55: 661-672.
44. Kupper H., Kupper F, Spiller M (1996) Environmental relevance of heavy metal substituted chlorophylls using the example of water plants. J Exp Bot 47: 259-266.
45. Lane T W, Morel F M (2000) A biological function for cadmium in marine diatoms. Proc. Natl. Acad. Sci. USA 97:4627-4631.
46. Lee, S. B., Byun, M. O. and Daniell, H. (2003) Accumulation of trehalose within transgenic chloroplasts confers drought tolerance. Molecular Breeding, 11(1), 1-13.
47. Lee, A., Lin Z.-Q, Pickering I. J. and Terry N. (2001) X-ray absorption spectroscopy study shows that the rapid selenium volatilizer, pickleweed (Salicornia bigelovii Torr.) reduces selenate to organic forms without the aid of microbes. Planta, 213,977-980.
48. Li, Z. S., Lu, Y. P., Zhen, R. G., Szczypka, M., Thiele, D. J. and Rea, P. A. (1997) A new pathway for vacuolar cadmium sequestration in Saccharomyces cerevisiae: YCF11-catalyzed transport of bis(glutathionate)cadmium. Proc. Natl. Acad. Sci., 94, 42-47.
49. Lin Z Q, Hansen D, Zayed A M, Terry N (1995) Biological selenium volatilization: Method of measurement under field conditions. J Environ Qualt 28 (1): 309-315.
50. Lin, Z. Q., de Souza, M., Pickering, I. J. and Terry N. (2002) Evaluation of the Macroalga, Muskgrass, for the Phytoremediation of Selenium-Contaminated Agricultural Drainage Water by Microcosms. J. Environ. Qual., 31, 2104-2110.
51. Lin, Z. Q., Schemenauer R. S., Cervinka V., Zayed A., Lee, A. and Terry, N. (2000). Selenium volatilization from a soil-plant treatment system for the remediation of contaminated water and soil in the San Joaquin Valley. J. Environ. Qual., 29, 1048-1056.
52. Maliga, P., Carrer, H., Kanevski, I., Staub, J. and Svab, Z. (1993) Plastid engineering in land plant: a conservative genome is open to change. Phil. Trans. R. Soc. Lond., 342, 203-208.
53. McBride K E, Svab Z, Schaaf D J, Hogen P S, Stalker D M, Maliga P (1995) Amplification of a chimeric Bacillus genes in chloroplasts leads to an extraordinary level of an insecticidal protein in tobacco. Bio/Technology 13: 362-365.
54. Meagher R B, Rugh C L (1997) Phytoremediation of heavy metal pollution: Ionic and methyl mercury. in Organization for Economic Cooperation and Development (OECD) Document: Biotechnology for Water Use and Conservation, Pp. 305-321. The Mexico '96 Workshop. OEC D, Paris.
55. Meagher R B (2000) Phytoremediation of toxic elemental and organic pollutants. Curr Opin Plant Biol 3: 153-162.
56. Meagher, R. and Rugh, C. L. (1996) Phytoremediation of heavy metal pollution: ionic and methyl mercury. In: OECD Biotechnology for Water Use and Conservation. Workshop Organization for Economic Co-Operation and Development, Cocoyoc, Mexico pp. 305-321.
57. Meagher, R. B., Rugh, C. L., Kandasamy, M. K., Gragson, G. and Wang, N. J. (2000) Engineered phytoremediation of mercury pollution in soil and water using bacterial genes. In Phytoremediation of Contaminated Soil and Water (Terry, N. and Bañuelos, G. eds), Ann Arbor Press, Inc, Berkeley, Calif., pp. 201-219.
58. Meharg A A, Macnair M R (1992) Suppression of the high affinity phosphate uptake system, a mechanism of arsenate tolerance in Holcus lanatus L. J Exp Bot 43:519-524.
59. Millan, F. A., Mingo-Castel, A., Miller, M. and Daniell, H. (2003) A chloroplast transgenic approach to hyper-express and purify human serum albumin, a protein highly susceptible to proteolytic degradation. Plant Biotechnol. J, 1(2), 71-97.
60. Munns, R. K. and Holland, D. C. (1971) Determination of mercury in fish by flamless atomic absorption. J. Assoc. Off. Anal. Chem., 54, 202-205.
61. Murashige T., Skoog, F. (1962). A revised medium for rapid growth and bioassays with tobacco tissue cultures. Physiologia Plantarum 15, pp. 473-497.
62. Murphy, A. and Taiz, L. (1995) A new vertical mesh transfer technique for metal-tolerance studies in Arabidopsis: ecotypic variation and copper-sensitive mutants. Plant Physiol. 108, 29-38.
63. Nakamura, K., Sakamoto, M., Uchiyama, H. and Yagi, O. (1990) Organomercurials-volatilizing bacteria in the mercury-polluted sediment of Minamata Bay, Japan. Appl. Environ. Microbiol., 56, 304-305.
64. Nies D H (1999) Microbial heavy metal resistance. Appl Microbiol Biotechnol 51: 730-750.
65. Noctor, G. and Foyer, C. H. (1998) Ascorbate and glutathione: keeping active oxygen under control. Annu. Rev. Plant Physiol. Plant. Mol. Biol., 49, 249-279.
66. Noctor, G., Arisi, A. C. M., Jouanin, L., Kuner, K. J., Rennenberg, H. and Foyer, C. (1998) Glutathione: biosynthesis: metabolism and relationship to stress tolerance explored in transformed plants. J. Exp. Bot. 49, 623-647.
67. Ogawa H I, Tolle C L, Summers A O (1984) Physical and genetic map of the organomercury resistance (Omr) and inorganic mercury resistance (Hgr) loci of the IncM plasmid R83 lb. Gene 32: 311-320.
68. Patra M, Sharma A (2000) Mercury toxicity in plants. Botanical Review 66 (3): 379-422
69. Pickering, I. J., G. E. Brown, Jr. and T. K. Tokunaga. (1995) Quantitative speciation of selenium in soils using X-ray absorption spectroscopy. Environ. Sci. Technol., 29, 2456-2459.
70. Pilon-Smits E A H, Pilon M (2000) Breeding mercury-breathing plants for environmental clean-up. Trends Plant Sci 5:235-236.
71. Pilon-Smits E. A., Hwang S., MeI Lytle, C., Zhu Y., Tai, J. C., Bravo, R. C., Chen, Y., Leustek, T. and Terry, N. (1999) Overexpression of ATP sulfurylase in Indian mustard leads to increased selenate uptake, reduction, and tolerance. Plant Physiol., 119, 123-132.
72. Prasad D D K, Prasad A R K (1987) Altered o-aminolevulinic acid metabolism by lead and mercury in germination seedlings of bajra (Pennisetum typoideum). J Plant Physiol 127: 241-249.
73. Riddle, S. G., Tran, H. H., Dewitt, J. G. and Andrews J. C. (2002). Field, Laboratory, and X-ray Absorption Spectroscopic Studies of Mercury Accumulation by Water Hyacinths. Environ. Sci. Technol., 36(9), 1965-1970.
74. Rinderle S J, Booth J E, Williams J W (1983) Mercuric reductase from R-plasmid NR1: characterization and mechanistic study. Biochemistry 22: 869-
75. Rugh C L, Senecoff, J F, Meagher R B, Merkle S A (1998) Development of transgenic yellow poplar for mercury phytoremediation. Nat Biotechnol 16: 925-928.
76. Rugh Clayton L, Wilde H. D, Stack N M, Thompson D M, Summers A 0, Meagher R B (1996) Mercuric ion reduction and resistance in transgenic Arabidopsis thaliana plants expressing a modified bacterial merA gene. In: Proceedings of the National Academy of Sciences of the United States of America. 93 (8): 3182-3187.
77. Rugh, C. L., Bizily, S. P. and Meagher, R. B. (1999) Phytoremediation of environmental mercury pollution. In Phytoremediation of Toxic Metals: Using Plants to Clean-up the Environment (Ensley, B. and Raskin, I. eds), John Wiley and Sons, New York, pp. 151-169.
78. Ruiz, O. N., Hussein H. S., Terry, N. and Daniell, H. (2003) Phytoremediation of organomercurial compounds via chloroplast genetic engineering. Plant Physiol., in press.
79. Salt D E, Smith R D, Raskin 1 (1995) Phytoremediation: A novel strategy for the removal of toxic metals from the environment using plants. Bio-Technology (New York) 13 (5): 468-474.
80. Sen A K, Mondal N G (1987) Silvinia natans as the scavenger of Hg (II). Water Air Soil Pollut 34: 439-446.
81. Sinha S, Gupta M, Chandra P (1996) Bioaccumulation and biochemical effects of mercury in the plant Bacopa monnieri (L). Environ. Toxicol Water Qualt 11: 105-112.
82. Staub J M, Garcia B, Graves J, Hajdukiewicz P T J, Hunter P, Nehra N, Paradkar V, Schlittler M, Carol J A, Spatola L, Ward D, Ye G, Rusell D (2000) High-yield production of human therapeutic protein in tobacco chloroplast. Nat Biotechnol 18, 333-338.
83. Sugita M, Sugiura M (1996) Regulation of genes expression in chloroplast of higher plants. Plant Mol Biol 32: 315-326.
84. Terry N, Zayed A M, De Souza M P, Tarun A S (2000) Selenium in higher plants. Ann. Rev. Plant Physiol Plant Mol Biol 51: 401-432.
85. Terry, N. and Zayed, A. M. (1994) Selenium volatilization by plants. In Selenium in the Environment (Frankenberger, Jr. W. T., Benson, S. eds), Marcel Dekker, New York, pp 343-369.
86. Terry, N. and Zayed, A. M. (1998) Phytoremediation of selenium. In Selenium in the Environment (Frankenberger, Jr. W. T., Benson, S. eds), Marcel Dekker, New York, pp 633-657.
87. Terry, N., Carlson, C., Raab, T. K. and Zayed, A. M. (1992) Rates of selenium volatilization among crop species. J. Environ. Qual., 21, 341-344.
88. USEPA [U. S. Environmental Protection Agency] (1984) Health effects assessment of mercury. Environmental Criteria and Assessment Office, Cincinnati, Ohio.
89. Weckx, J. E. J. and Clijsters, H. M. M. (1996) Oxidative damage and defense mechanisms in primary leaves of Phaseolus vulgaris as a result of root assimilation of toxic amounts of copper. Physiol Plant., 96, 506-512.
90. Weckx, J. E. J. and Clijsters, H. M. M. (1997) Zn phytotoxicity induces oxidative stress in primary leaves of Phaseolus vulgaris. Plant Physiol. Biochem., 35, 405-410.
91. Zayed, A., Lytle, C. M. and Terry N. (1998) Accumulation and volatilization of different chemical species of selenium by plants. Planta 206, 284-292.
92. Zayed, A. M. and Terry, N, (1992) Selenium volatilization in broccoli as influenced by sulfate supply. J Plant. Physiol., 140, 646-652.
93. Zayed, A. M. and Terry, N. (1994) Selenium volatilization in roots and shoots: effects of shoot removal and sulfate level. J. Plant Physiol. 143, 8-14.
94. Zhu, Y. L., Pilon-Smits E. A. H., Tarun, A. S., Weber, U. S., Jouanin L. and Terry N. (1999) Cadmium Tolerance and Accumulation in Indian Mustard Is Enhanced by Overexpressing γ-Glutamylcysteine Synthetase. Plant Physiol., 121, 1169-1177.

1. A plastid transformation vector for stably transforming a plastid genome, comprising, as operably-linked components, a first flanking sequence, at least one DNA sequence coding for a polypeptide suitable for remediating a contaminant compound, and a second flanking sequence, wherein a plant is stably transformed with said plastid transformation vector, and said plant is capable of phytoremediating a comtaminant compound.

2. The vector of claim 1, wherein said at least one DNA sequence coding for a polypeptide suitable for remediating a contaminant compound is a phtyoremediation operon.

3. The vector of claim 1 or 2 further comprising a regulatory sequence.

4. The vector of claim 3, wherein said regulatory sequence comprises a promoter operative in said plastid genome.

5. The vector of claim 4, wherein said promoter is 16srRNA.

6. The vector of claim 3, wherein said regulatory sequence comprises a 3′untranslated region (UTR).

7. The vector of claim 1, wherein the vector is competent for stabling integrating in the plastid genome of a plant species and wherein the flanking DNA sequences are substantially homologous to sequences in a spacer region of said plastid genome, and wherein said flanking sequences are conserved in the plastid genome of said plant species.

8. The vector of claim 7, wherein said spacer region is a transcriptionally active spacer region.

9. The vector of claim 1, wherein the plastid genome is selected from the group consisting of chloroplast, chromoplast, amyloplast, proplastide, leucoplast and etioplast.

10. The vector of claim 1 further comprising a DNA sequence encoding a selectable marker encoding an antibiotic-free selectible marker.

11. The vector of claim 1, wherein said first flanking sequence is trnI, and wherein said second flanking sequence is trnA.

12. The vector of claim 11, wherein trnI and trnA provide for homologous recombination to insert an operon coding for a protein suitable for inactivating a contaminant compound into the spacer region in an inverted repeat region of a chloroplast genome.

13. The vector of claim 1, wherein said operon is located in a single copy region of said plastid genome.

14. The vector of claim 6, wherein said 3′UTR is a 3′UTR of psbA.

15. The vector of claim 1, further comprising a DNA sequence encoding a selectable marker.

16. The vector of claim 15, wherein said selectable marker is an antibiotic-free selectable marker.

17. The vector of claim 16, wherein said antibiotic-free selectable marker is Betaine aldehyde dehydrogenase (BADH).

18. The vector of claim 15, wherein said DNA sequence encoding a selectable marker encodes an antibiotic resistant selectable marker.

19. The vector of claim 18, wherein said antibiotic resistant selectable marker is aadA.

20. A method for producing at least one DNA sequence coding for a protein suitable for inactivating a contaminant compound comprising:
integrating the plastid transformation vector of claim 1 into the plastid genome of a plant cell;
growing said plant cell to thereby express said at least one heterologous DNA sequence coding for a protein suitable for inactivating a contaminant compound.

21. The method of claim 20, wherein said at least one DNA sequence coding for a protein suitable for inactivating a contaminant compound is competent to phytoremediate a contaminant compound.

22. A plant stably transformed with the transformation vector of claim 1.

23. A progeny of the plant of claim 22.

24. A seed of the plant of claim 22.

25. A plant part of the plant of claim 22, comprising a plastid including said at least one heterologous DNA sequence coding for a protein suitable for inactivating a contaminant compound

26. The plant of claim 22, wherein said plant further comprises at least one chloroplast transformed with the vector of claim 1.

27. The plant of claim 22, wherein said plant further comprises mature leaves transformed with the vector of claim 1.

28. The plant of claim 22, wherein said plant further comprises young leaves
transformed with the vector of claim 1.

29. A plastid transformation vector for stably transforming a plastid genome, comprising, as operably-linked components, a first flanking sequence capable for integrating said plastid transformation vector into the plastid genome, an operon comprising merA and merB genes, and a second flanking sequence capable for integrating said plastid transformation vector into the plastid genome.

30. The plastid transformation vector of claim 29, wherein said first and second flanking sequences allow site-specific integration of the operon containing the merA and merB genes into an inverted repeat region of the plastid genome between trnI (tRNA Ile) and trnA (tRNA Ala) genes.

31. The plastid transformation vector of any one of claims 29 or 30, wherein said operon further comprises the aadA gene.

32. The plastid transformation vector of any one of claims 29-31, further comprising a 3′ untranslated region (3′UTR) positioned downstream of the operon, and upstream of said second flanking sequence.

33. The plastid transformation vector of claim 32, wherein said 3′UTR is from a psbA chloroplast gene

34. A method of detoxifying mercury comprising the steps of:
integrating the vector of claim 45 into a plastid genome of a plant cell culturing said plant cell to express merA and merB,
exposing said plant cells to mercury.

35. The vector of claim 2 wherein the operon is the merAB operon.

36. A plant cell comprising a plastid including an expression cassette, said expression cassette comprising as operably linked components, a promoter functional in said plastid, an operon encoding a merAB operon, a transcription termination region, and DNA sequences flanking the expression cassette to facilitate stable integration of said expression cassette into a genome of said plastid by homologous recombination.

37. A plant cell comprising a plastid including an expression cassette, said expression cassette comprising as operably linked components, a promoter functional in said plastid, an operon encoding a phytoremediation operon, a transcription termination region, and DNA sequences flanking the expression cassette to facilitate stable integration of said expression cassette into a genome of said plastid by homologous recombination.

38. The plastid transformation vector of claim 1, wherein said at least one DNA sequence coding for a polypeptide suitable for remediating a contaminant compound is a bacterial gene onr, encoding PETN reductase.

39. The plastid transformation vector of claim 1, wherein said at least one DNA sequence coding for a polypeptide suitable for remediating a contaminant compound is a cytochrome P450.

40. The plastid transformation vector of claim 1, wherein said at least one DNA molecule coding for a polypeptide suitable for remediating a contaminant compound is a gene coding for phytochelatin synthase, wherein said phytochelatin synthase enables a trnsformed plant to sequester heavy metal ions.
